(19) United States
(12) Patent Application Publication
(10) Pub. No.: US 2023/0042936 A1
(43) Pub. Date: Feb. 9, 2023

(54) PYRIDO[2,3-d]PYRIMIDIN-7(8H)-ONES AS CDK INHIBITORS

(71) Applicants: PROSENESTAR LLC, New York, NY (US); Fundacion del sector publico estatal centro nacional de investigaciones oncologicas Carlos III (F.S., Madrid (ES)

(72) Inventors: Marcos Malumbres, New York, NY (US); Mónica Alvarez-Fernández, New York, NY (US); Elisabet Zapatero, New York, NY (US); María Salazar-Roa, New York, NY (US); Juan Sanchez, New York, NY (US)

(21) Appl. No.: 17/942,508

(22) Filed: Sep. 12, 2022

Related U.S. Application Data
(63) Continuation of application No. PCT/US2021/022328, filed on Mar. 15, 2021.
(60) Provisional application No. 62/989,448, filed on Mar. 13, 2020.

(51) Int. Cl. C07D 471/04; A61P 35/00

(52) U.S. Cl. CPC C07D 471/04; A61P 35/00

(57) ABSTRACT

The pyrido[2,3-d]pyrimidin-7(8H)-ones of Formula 1 and pharmaceutical compositions containing compounds of Formula 1 as CDK inhibitors are disclosed herein. Methods and use of a compound of Formula 1 in the treatment of cancer and manufacture are also disclosed.

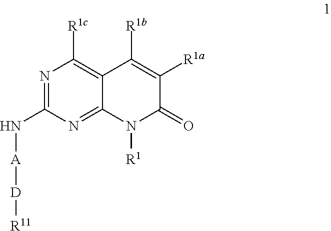

### CROSS-REFERENCE TO RELATED APPLICATIONS

This application is a continuation of International Pat. App. No. PCT/US2021/022328, filed Mar. 15, 2021; which claims the benefit of U.S. provisional patent application No. 62/989,448, filed Mar. 13, 2020; all of which are incorporated by reference in their entirety.

### BACKGROUND

Cyclin-dependent kinases (CDK) inhibitors have therapeutic potential for several diseases including cancer, diabetes, renal, neurodegenerative and infectious diseases. However, the focus has been on their development as anticancer drugs, with emphasis on the cell cycle and transcriptional CDKs.

Cancer represents a pathological manifestation of uncontrolled cell division. Therefore, it has long been anticipated that our understanding of the basic principles of cell cycle control would result in effective cancer therapies. In particular, CDKs that promote transition through the cell cycle, such as CDK4 and CDK6, were expected to be key therapeutic targets because many tumorigenic events ultimately drive proliferation by activating these kinases in the G1 phase of the cell cycle thus triggering DNA synthesis (S-phase). Moreover, perturbations in genomic stability during S-phase or mitosis (M), two phases regulated by CDK2 and CDK1, are pivotal tumorigenic events. CDK4 and CDK6 are considered highly validated anticancer drug targets due to their essential role regulating cell cycle progression during the G1-to-S-phase transition. However, translating this knowledge into successful clinical development of CDK inhibitors has historically been challenging. For example, CDK4 and CDK6 suppression seems to have little clinical effect in certain types of cancer such as colorectal cancer, triple-negative breast cancer and melanomas. Therefore, searching for a more effective CDK inhibitor with broader spectrum in treating cancer with low toxicity continues.

### SUMMARY

Described herein are pyrido[2,3-d]pyrimidin-7(8H)-one compounds, pharmaceutically acceptable salts, solvates, prodrug and active metabolites, that can be potent CDK2, CDK4, and CDK6 inhibitors. These compounds may be used to treat various types of cancer in need thereof comprising administering a therapeutically effective amount of a pyrido[2,3-d]pyrimidin-7(8H)-one compound.

Some embodiments include a compound represented by Formula 1:

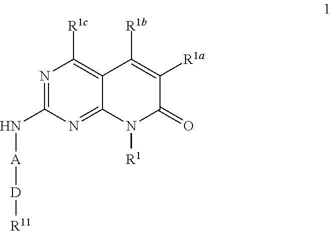

wherein R¹ is optionally substituted C₁₋₆ alkyl or optionally substituted C₃₋₁₀ cycloalkyl; R^{1a} is R⁸, - or COR⁸; R^{1b} is R⁸; R^{1c} is R⁸, CN, —OR⁸, NR⁹R⁸, optionally substituted C₆₋₁₀ aryl or optionally substituted C₁₋₁₀ heteroaryl; A is optionally substituted aryl or optionally substituted heteroaryl; D is optionally substituted piperidin-1,4-yl or is optionally substituted piperazin-1,4-yl; R¹¹ is R⁸, —OR⁸, SO₂R⁸, SO₂NR⁸R⁹, COR⁸, CO₂R⁸, or CONR⁸R⁹, wherein R⁸ and R⁹ are independently H, or C₁₋₆ hydrocarbyl optionally substituted with F, Cl, Br, I, amino, OH, C₁₋₆—O-alkyl, cyano, or a C₁₋₆ geminal -alkyl-O-alkyl-; such as a ring.

Some embodiments include a subject composition, which is a composition comprising a subject compound. A subject compound is a compound described herein, such as a compound of formula 1, Formula 1A, Formula 1B, Formula 1C, Formula 2, Formula 2A, Formula 3, Formula 3A, Formula 4, Formula 4A, Formula 5, Formula 5A, Formula 6, Formula 6A, Formula 7, Formula 7A, Formula 8, or Formula 9, or a pharmaceutically acceptable salt, hydrate, tautomer, or stereoisomer thereof.

Some embodiments include a pharmaceutical dosage form comprising a subject compound.

A subject compound or a subject composition may be used for inhibiting CDK2, CDK4 and/or CDK6, or for treating cancer. A subject compound or a subject composition may also be used for treating disease or disorder such as breast cancer, melanoma, renal cancer, squamous cell carcinoma, bladder cancer, pancreatic cancer, ovarian cancer, lung cancer, prostate cancer, colon cancer, oesophageal cancer, head cancer, neck cancer, neuroblastoma, myeloma, glioma, lymphomas and leukemias.

Some embodiments include a method of treating a disease or disorder associated with CDK2, CDK4 and/or CDK6 inhibitors comprising administering an effective amount of a subject compound to a mammal in need thereof.

Some embodiments include use of a subject compound in the manufacture of a medicament for the treatment of a disease or disorder associated with a CDK2, CDK4 or CDK6 inhibitor.

### BRIEF DESCRIPTION OF THE FIGURES

FIG. 1 depicts DNA content profiles of MDA-MB-231 human breast cancer cells treated with subject compounds or commercial CDK inhibitors, showing the percentage of cells at the different cell cycle phases after 48 hours of the treatments indicated. (n=6 technical replicates).

FIG. 2 depicts B-Galactosidase staining in MDA-MB-231 human breast cancer cells treated with available CDK inhibitors or subject compounds, with representative pictures of 3-Gal activity after 14 day-treatment (arrows point to positive-stained cells).

FIG. 3 depicts the results of B-Galactosidase staining in MDA-MB-231 human breast cancer cells treated with available CDK inhibitors or subject compounds showing the quantification of percentage of β-Galactosidase positive cells (indicated as blue columns) and total cell number (shown as black dots) at day 3 and day 14 after treatment with the indicated compounds (n=2 independent experiments).

FIG. 4 depicts specific effect of selected subject compounds in Cdk-deficient mouse embryonic fibroblasts (MEFs). Relative cell count at day 6 versus day 3 in Cdk2-, Cdk 4/6- or Cdk2/4/6-null MEFs after the indicated treatments. Data are mean±s.e.m. (3 technical replicates).

FIG. 5 depicts cell growth of luminal-like and non-luminal breast cancer cell lines in the presence of palbociclib or PS009. Relative cell count at day 6 after starting the treatment with palbociclib or PS009 (G150 dose in each case), in a group of luminal-like (ZR75-1, T47D, MCF7) and non-luminal (HCC1143, MDA-MB-231, BT549, MDA-MB468) breast cancer cell lines. Data are mean±s.e.m. (3 technical replicates). Note that among the non-luminal breast cancer cell lines, Palbociclib exhibits an obvious pRB dependence whereas PS009 is efficient in both pRB-wild-type and mutant cell lines.

FIG. 6 shows effect of subject compounds on different cell cycle markers. Biochemical analysis of pRB-proficient and pRB-deficient breast cancer cells treated with the indicated compounds at the corresponding GI₅₀ dose. Actin was used as a loading control. Blots shown are representative of more than 3 independent experiments.

FIG. 7 depicts the effect of PS004, PS006 and PS009 on the phosphorylation of the retinoblastoma protein in different tissues using antibodies against phospho RB1 Ser807/811. Micrographs are representative from three different mice per treatment.

FIG. 8A depicts the therapeutic effect of subject compounds in xenotransplants with MDA-MB-231 breast cancer cells. Mice harboring MDA-MB-231-derived xenografts were treated with the compounds indicated during 2 weeks (4 total doses per treatment). Tumor weight (g) was measured at the endpoint of the different treatments. DMSO treatment is the control for PS compounds, administered intraperitoneally and lactate buffer is the control for Palbociclib, administered orally. Data are mean±s.e.m. (every dot represents one mouse analyzed). **P<0.01; ***P<0.001 (Student's t-test).

FIG. 8B depicts the therapeutic effect of subject compounds in xenotransplants with MDA-MB-231 breast cancer cells for time-lapse analysis of tumor fold growth after treatment with the indicated compounds. Mice harboring MDA-MB-231-derived xenografts were treated with the compounds indicated during 2 weeks (4 total doses per treatment). The mean of the DMSO treatment and lactate buffer shown in FIG. 8A at every time point is represented as “vehicle”. Data are mean±s.e.m. (every dot represents one mouse analyzed). **P<0.01; ***P<0.001 (Student's t-test).

FIG. 9A depicts the quantification of cells positive for the phosphorylated form of RB1 (pRb) in the tumors analyzed. Data are mean±s.e.m. (n=6 mice per treatment). *P<0.05;***P<0.001 (Student's t-test).

FIG. 9B depicts the quantification of cells positive for the presence of Ki67 in the tumors analyzed. Data are mean±s.e.m. (n=6 mice per treatment). *P<0.05;***P<0.001 (Student's t-test).

FIG. 10 depicts the effect of treatment of mice with different compounds on different parameters including total mouse weight and the number of different cell populations such as red blood cells or different white blood cell populations in peripheral blood. Data correspond to 6 mice per treatment and 13 control mice.

FIG. 11 depicts representative micrographs of lung, bone marrow, and intestine sections after treatment of mice with the indicated compounds. Sections were stained with hematoxylin and eosin. Samples are representative of at least 3 mice per treatment.

### DETAILED DESCRIPTION

Unless otherwise indicated, when a compound or chemical structural feature, such as pyrido[2,3-d]pyrimidin-7(8H)-one, aryl, heteroaryl, etc., is referred to as being “optionally substituted,” it includes a feature that has no substituents (i.e. unsubstituted), or a feature that is “substituted,” meaning that the feature has one or more substituents. The term “substituent” has the broadest meaning known to one of ordinary skill in the art and includes a moiety that replaces one or more hydrogen atoms attached to a parent compound or structural feature. In some embodiments, a substituent may be an ordinary moiety of any organic compound known in the art, which may have a molecular weight (the sum of the atomic masses of the atoms of the substituent) of 15 Da to 50 Da, 15 Da to 100 Da, 15 Da to 150 Da, 15 Da to 200 Da, 15 Da to 300 Da, or 15 Da to 500 Da. In some embodiments, a substituent comprises, or consists of: 0-30, 0-20, 0-10, or 0-5 carbon atoms; and 0-30, 0-20, 0-10, or 0-5 heteroatoms, wherein each heteroatom may independently be: N, O, S, P, Si, F, Cl, Br, or I; provided that the substituent includes one C, N, O, S, P, Si, F, Cl, Br, or I atom. Examples of substituents include, but are not limited to, hydrocarbyl, such as alkyl, alkenyl, alkynyl, aryl, etc.; heterohydrocarbyl, such as heteroalkyl, heteroalkenyl, heteroalkynyl, heteroaryl, alkoxy, aryloxy, acyl, acyloxy, alkylcarboxylate, alkylthio, thiocarbonyl, O-carbamyl, N-carbamyl, O-thiocarbamyl, N-thiocarbamyl, C-amido, N-amido, S-sulfonamido, N-sulfonamido, isocyanato, thiocyanato, isothiocyanato, haloalkyl, haloalkoxyl, trihalomethanesulfonyl, trihalomethanesulfonamido, etc.; or other O, S, N, Si, P, or halo-based substituents that are not necessarily hydrocarbyl or heterocarbyl, such as hydroxy, thiol, cyano, F, Cl, Br, I, nitro, silyl, sulfenyl, sulfinyl, sulfonyl, amino, phosphono, phosphoric acidyl, etc.

For convenience, the term “molecular weight” is used with respect to a moiety or part of a molecule to indicate the sum of the atomic masses of the atoms in the moiety or part of a molecule, even though it may not be a complete molecule.

The structures associated with some of the chemical names referred to herein are depicted below. These structures may be unsubstituted, as shown below, or a substituent may independently be in any position normally occupied by a hydrogen atom when the structure is unsubstituted. Unless a point of attachment is indicated by

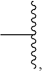

attachment may occur at any position normally occupied by a hydrogen atom.

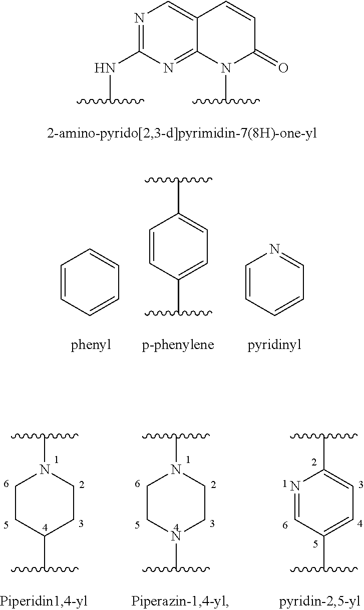

As used herein, the term “alkyl” has the broadest meaning generally understood in the art and may include a moiety composed of carbon and hydrogen containing no double or triple bonds. Alkyl may be linear alkyl, branched alkyl, cycloalkyl, or a combination thereof and in some embodiments, may contain from one to thirty-five carbon atoms. In some embodiments, alkyl may include C₁₋₁₀ linear alkyl, such as methyl (—CH₃), methylene (—CH₂—), ethyl (—CH₂CH₃), ethylene (—C₂H₄—), propylene (—C₃CH₆—), n-butyl (—CH₂CH₂CH₂CH₃), n-pentyl (—CH₂CH₂CH₂CH₂CH₃), n-hexyl (—CH₂CH₂CH₂CH₂CH₂CH₃), etc.; C₃₋₁₀ branched alkyl, such as C₃H₇ (e.g. iso-propyl), C₄H₉ (e.g. branched butyl isomers), C₅H₁₁ (e.g. branched pentyl isomers), C₆H₁₃ (e.g. branched hexyl isomers), C₇H₁₅ (e.g. heptyl isomers), etc.; C₃₋₁₀ cycloalkyl, such as C₃H₅ (e.g. cyclopropyl), C₄H₇ (e.g. cyclobutyl isomers such as cyclobutyl, methylcyclopropyl, etc.), C₅H₉ (e.g. cyclopentyl isomers such as cyclopentyl, methylcyclobutyl, dimethylcyclopropyl, etc.) C₆H₁₁ (e.g. cyclohexyl isomers), C₇H₁₃ (e.g. cycloheptyl isomers), etc.; and the like.

As used herein the term “aryl” has the broadest meaning generally understood in the art and may include an aromatic ring or aromatic ring system such as phenyl, naphthyl, etc.

The term “heteroaryl” also has the meaning understood by a person of ordinary skill in the art and includes an “aryl” which has one or more heteroatoms in the ring or ring system, such as pyridinyl, furyl, thienyl, oxazolyl, thiazolyl, imidazolyl, triazolyl, oxadiazolyl, isoxazolyl, indolyl, quinolinyl, benzofuranyl, benzothienyl, benzooxazolyl, benzothiazolyl, benzoimidazolyl, etc.

Unless otherwise indicated, any reference to a compound herein by structure, name, or any other means includes pharmaceutically acceptable salts, such as HCl, HBr, HI, H₂SO₄, acetate, citrate, sodium, potassium, and ammonium salts; prodrugs, such as ester prodrugs; alternate solid forms, such as polymorphs, solvates, hydrates, etc.; tautomers; or any other chemical species that may rapidly convert to a compound described herein under conditions in which the compounds are used as described.

If stereochemistry is not indicated, a name or structural representation includes any stereoisomer or any mixture of stereoisomers.

Some of the embodiments include a compound represented by 1A, 1B, 1C, 2, 2A, 3, 3A, 4, 4A, 5, 5A, 6, 6A, 7, 7A, 8, or 9.

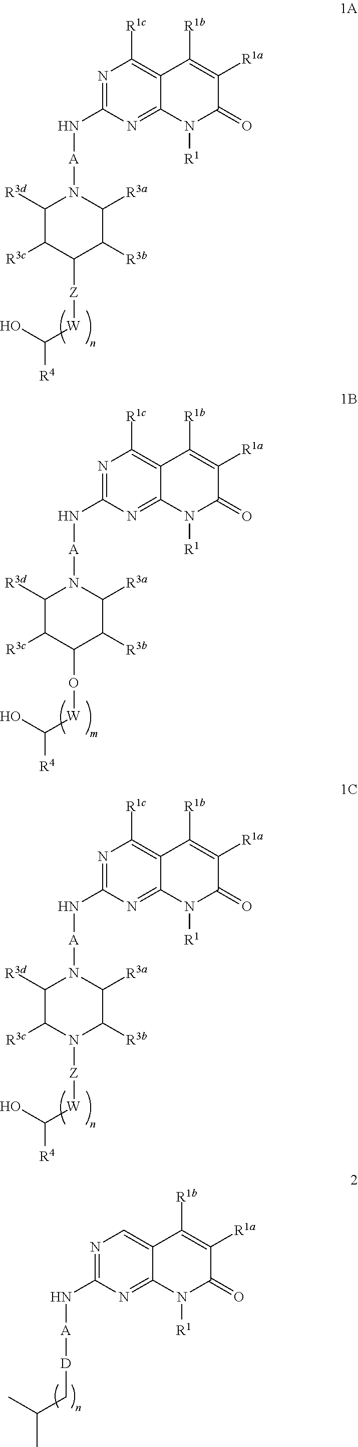

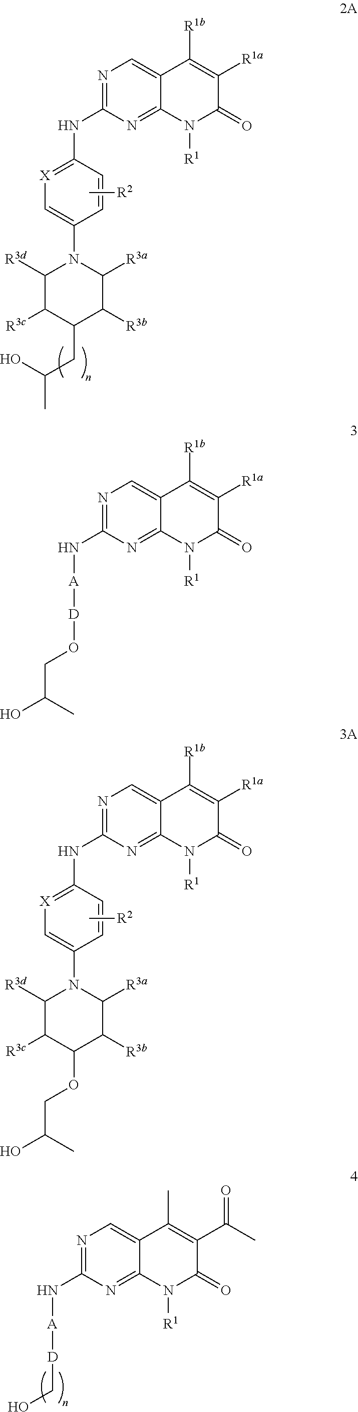

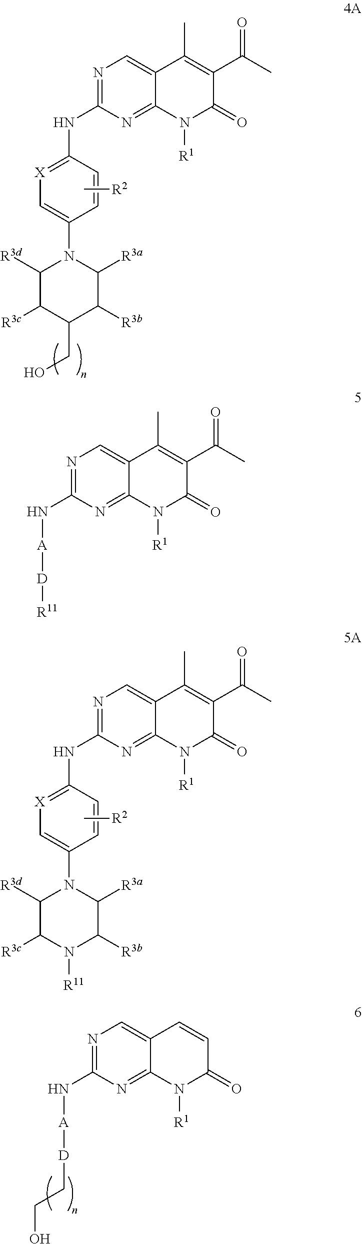

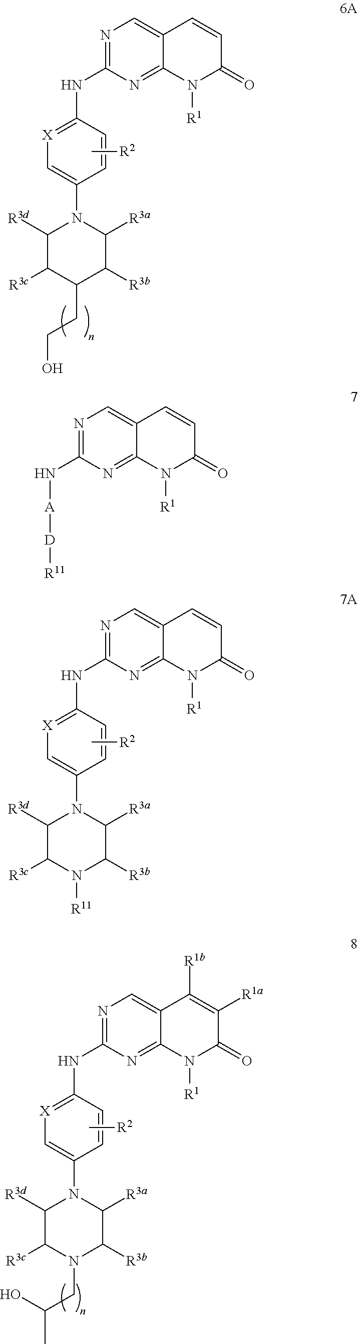

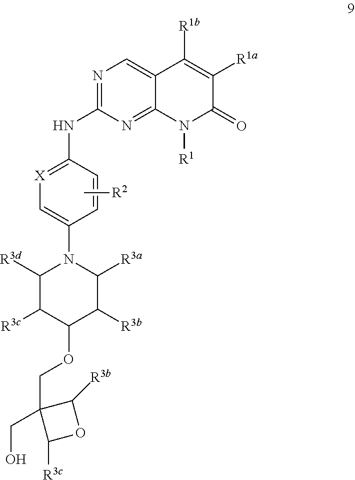

With respect to any relevant structural representation, such as Formula I, 1A, 1B or 1C, 2, 3, 4, 5, 6, 7, A is optionally substituted aryl or heteroaryl. In some embodiments, A is optionally substituted aryl, such as optionally substituted p-phenylene. In some embodiments, A is unsubstituted aryl. In some embodiments, A is optionally substituted heteroaryl. In some embodiments, A is unsubstituted heteroaryl. If the aryl or heteroaryl is substituted, it may have 1, 2, 3, or 4 substituents, wherein each substituent can be the same or different from the other substituents. Any substituent may be included on the aryl or heteroaryl. In some embodiments, some or all of the substituents on the aryl or heteroaryl may have: from 0 to 10 carbon atoms and from 0 to 10 heteroatoms, wherein each heteroatom is independently: O, N, S, F, Cl, Br, or I; and/or a molecular weight of 15 g/mol to 500 g/mol. In some embodiments, some or all of the substituents may each have a molecular weight of 15 Da to 200 Da, 15 Da to 100 Da, or 15 Da to 50 Da, and consist of 2 to 5 chemical elements, wherein the chemical elements are independently C, H, O, N, S, F, Cl, or Br.

For example, with respect to any relevant structural representation, such as Formula I, 1A, 1B or 1C, 2, 3, 4, 5, 6, 7, the substituents of A may be C₁-10 optionally substituted alkyl, such as CH₃, C₂H₅, C₃H₇, cyclic C₃H₅, C₄H₉, cyclic C₄H₇, C₅H₁₁, cyclic C₅H₉, C₆H₁₃, cyclic C₆H₁₁, etc., which may be optionally substituted; C₁₋₁₀ optionally substituted alkoxy such as OCH₃, OC₂H₅, OC₃H₇, cyclic OC₃H₅, OC₄H₉, cyclic OC₄H₇, OC₅H₁₁, cyclic OC₅H₉, OC₆H₁₃, cyclic OC₆H₁₁, etc.; halo, such as F, Cl, Br, I; OH; CN; NO₂; C₁₋₆ fluoroalkyl, such as CF₃, CF₂H, C₂F5, etc.; C₁₋₆ fluoroalkoxy, such as OCF₃, OCF₂H, OC₂F5, etc.; a C₁-10 ester such as —O₂CCH₃, —CO₂CH₃, —O₂CC₂H₅, —CO₂C₂H₅, —O₂C— phenyl, —CO₂-phenyl, etc.; a C₁₋₁₀ ketone such as —COCH₃, —COC₂H₅, —COC₃H₇, —CO-phenyl, etc.; or a C₁₋₁₀ amine such as NH₂, NH(CH₃), N(CH₃)₂, N(CH₃)C₂H₅, etc. In some embodiments, a substituent of A may be F, Cl, Br, I, CN, NO₂, C₁₋₄ alkyl, C₁₋₄ alkyl-OH, C₁₋₃ O-alkyl, CF₃, C(O)H, C₁₋₄ CO-alkyl, CO₂H, C₁₋₄ CO₂-alkyl, NH₂, or C₁₋₄ alkylamino.

With respect to any relevant structural representation, such as Formula 1, 1A, 1B, 1C, 2, 3, 4, 5, 6 or 7, in some embodiments A is optionally substituted p-phenylene, or optionally substituted pyridin-2,5-yl; wherein the 2-position attaches to NH and the 5-position attaches to D.

With respect to any relevant structural representation, such as Formula 1, 1A, 1B, 1C, 2, 3, 4, 5, 6 or 7, if A is a substituted phenylene, it may have 1, 2, 3, or 4 substituents, e.g. as represented in the structure below, wherein R^{2a}, R^{2b}, R^{2c} and R^{2d} are not all H.

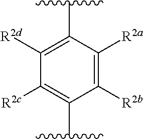

In some embodiments, A is unsubstituted p-phenylene,

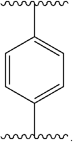 [SMILES: *c1ccc(*)cc1]

With respect to any relevant structural representation, such as Formula 1, 1A, 1B, 1C, 2, 3, 4, 5, 6 or 7, in some embodiments, A is fluoro-p-phenylene,

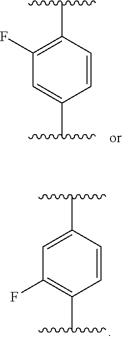 [SMILES: *c1ccc(*)c(F)c1 ; *c1ccc(*)c(F)c1]

With respect to any relevant structural representation, such as 1, 1A, 1B, 1C, 2, 3, 4, 5, 6 or 7, in some embodiments, A is optionally substituted pyridinyl, such as optionally substituted pyridin-2,5-yl. In some embodiments, A is unsubstituted pyridinyl. With respect to any relevant structural representation, such as Formula 1, 1A, 1B, 1C, 2, 3, 4, 5, 6 or 7, in some embodiments, A is unsubstituted 2-pyridinyl,

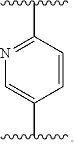 [SMILES: *c1ccc(*)nc1]

With respect to any relevant structural representation, such as Formula 1A, 1B, 1C, 2A, 3A, 4A, 5A, 6A, 7A, 8, or 9, in some embodiments, D is optionally substituted piperidin-1,4-yl or optionally substituted piperazin-1,4-yl. In some embodiments, D is optionally substituted piperazin-1,4-yl. In some embodiments, D is optionally substituted piperidin-1,4-yl. In some embodiments, D is optionally substituted piperidin-1,4-yl, wherein the 1-position attaches to A.

If D is substituted piperidin-1,4-yl, or substituted piperazin-1,4-yl, it may have 1, 2, 3, 4, 5, 6, 7 or 8 substituents, wherein each substituent can be the same or different from the other substituents. In some embodiments, some or all of the substituents of D may have from 0 to 10 carbon atoms and from 0 to 10 heteroatoms, wherein each heteroatom is independently O, N, S, F, Cl, Br, or I (provided that there is at least 1 non-hydrogen atom); and/or a molecular weight of 15 g/mol to 500 g/mol. In some embodiments, some or all of the substituents may each have a molecular weight of 15 Da to 200 Da, 15 Da to 100 Da, or 15 Da to 50 Da, and consist of 2 to 5 chemical elements, wherein the chemical elements are independently C, H, O, N, S, F, Cl, or Br.

For example, with respect to any relevant structural representation, such as Formula 1A, 1B, 1C, 2A, 3A, 4A, 5A, 6A, 7A, 8, or 9, the substituents of D may be optionally substituted alkyl, such as CH₃, C₂H₅, C₃H₇, cyclic C₃H₅, C₄H₉, cyclic C₄H₇, C₅H₁₁, cyclic C₅H₉, C₆H₁₃, cyclic C₆H₁₁, etc; C₁₋₁₀ optionally substituted alkoxy such as OCH₃, OC₂H₅, OC₃H₇, cyclic OC₃H₅, OC₄H₉, cyclic OC₄H₇, OC₅H₁₁, cyclic OC₅H₉, OC₆H₁₃, cyclic OC₆H₁₁, etc.; halo, such as F, Cl, Br, I; OH; CN; NO₂; C₁₋₆ fluoroalkyl, such as CF₃, CF₂H, C₂F5, etc.; C₁₋₆ fluoroalkoxy, such as OCF₃, OCF₂H, OC₂F₅, etc.; C₁₋₁₀ ester such as —O₂CCH₃, —CO₂CH₃, —OCOC₂H₅, —CO₂C₂H₅, —OCO-phenyl, —CO₂-phenyl, etc.; C₁₋₁₀ ketone such as —COCH₃, —COC₂H₅, —COC₃H₇, —CO-phenyl, etc.; or C₁₋₁₀ amine such as NH₂, NH(CH₃), N(CH₃)₂, N(CH₃)C₂H₅, etc.

With respect to any relevant structural representation, such as Formula 1A, 1B, 1C, 2A, 3A, 4A, 5A, 6A, 7A, 8, or 9, in some embodiments, D is:

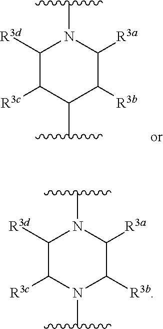

With respect to any relevant structural representation, such as Formula 1A, 1B, 1C, 2A, 3A, 4A, 5A, 6A, 7A, 8, or 9, in some embodiments, D is optionally substituted piperidin-1,4-yl. In some embodiments, D is unsubstituted piperidin-1,4-yl:

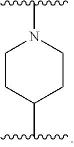 [SMILES: *C1CCN(*)CC1]

With respect to any relevant structural representation, such as Formula 1A, 1B, 1C, 2A, 3A, 4A, 5A, 6A, 7A, 8, or 9, in some embodiments, D is optionally substituted piperazine-1,4-yl. In some embodiments, D is unsubstituted piperazine-1,4-yl:

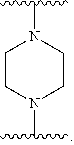 [SMILES: *N1CCN(*)CC1]

With respect to any relevant structural representation, such as Formula 3 or 4, A is optionally substituted p-phenylene, or optionally substituted pyridin-2,5-yl; wherein the 2-position attaches to NH and the 5-position attaches to D; and D is optionally substituted piperidin-1,4-yl, wherein the 1-position attaches to A.

With respect to any relevant structural representation, such as Formula 5, A is optionally substituted p-phenylene; and D is optionally substituted piperazin-1,4-yl.

With respect to any relevant structural representation, such as Formula 6, A is substituted p-phenylene, or optionally substituted pyridin-2,5-yl wherein the 2-position attaches to NH and the 5-position attaches to D; and D is optionally substituted piperidin-1,4-yl wherein the 1-position attaches to A.

With respect to any relevant structural representation, such as Formula 7, A is optionally substituted pyridin-2,5-yl wherein the 2-position attaches to NH and the 5-position attaches to D; and D is optionally substituted piperazin-1,4-yl; or A is optionally substituted phenyl and D is unsubstituted piperazin-1,4-yl.

With respect to any relevant structural representation, such as Formula 1, 1A, 1B, 1C, 2, 2A, 3, 3A, 4, 4A, 5, 5A, 6, 6A, 7, 7A, 8, or 9, generally R¹⁻²⁸, may be H or any substituent, such as a substituent having 0 to 12 atoms or 0 to 10 carbon atoms and 0 to 5 heteroatoms, wherein each heteroatom is independently: O, N, S, F, Cl, Br, or I, and/or having a molecular weight of 15 g/mol to 300 g/mol. Any of R¹⁻²⁸ may comprise: a) 1 or more alkyl moieties optionally substituted with, or optionally connected by or to, b) 1 or more functional groups, such as C═C, C≡C, CO, CO₂, CON, NCO₂, OH, SH, O, S, N, N=C, F, Cl, Br, I, CN, NO₂, CO₂H, NH₂, etc.; or may be a substituent having no alkyl portion, such as F, Cl, Br, I, NO₂, CN, NH₂, OH, COH, CO₂H, etc. In some embodiments, each of R¹⁻²⁸ is independently H, F, Cl, Br, I, or a substituent having a molecular weight of 15 Da to 300 Da, 15 Da to 200 Da, 15 Da to 100 Da, or 15 Da to 60 Da, and consisting of 2 to 5 chemical elements, wherein the chemical elements are independently C, H, O, N, S, F, Cl, or Br.

With respect to any relevant structural representation, such as Formula 1, 1A, 1B, 1C, 2, 2A, 3, 3A, 4, 4A, 5, 5A, 6, 6A, 7, 7A, 8, or 9, some non-limiting examples of R¹⁻²⁸ may include R^{A}, F, Cl, Br, CN, OR^{A}, C₁₋₃ fluoroalkyl, C₁₋₄ hydroxyalkyl, NO₂, NR^{A}R^{B}, COR^{A}, CO₂R^{A}, OCOR^{A}, NR^{A}COR^{B}, CONR^{A}R^{B}, etc. In some embodiments, R¹⁻²⁸ may be H; F; Cl; Br; CN; C₁₋₃ fluoroalkyl, such as CHF₂, CF₃, etc; OH; NH₂; C₁₋₆ alkyl, such as methyl, ethyl, propyl isomers (e.g. n-propyl and isopropyl), cyclopropyl, butyl isomers, cyclobutyl isomers (e.g. cyclobutyl and methylcyclopropyl), pentyl isomers, cyclopentyl isomers, hexyl isomers, cyclohexyl isomers, etc.; C₁₋₆ alkoxy, such as —O— methyl, —O-ethyl, isomers of —O-propyl, —O-cyclopropyl, isomers of —O-butyl, isomers of —O-cyclobutyl, isomers of —O-pentyl, isomers of —O-cyclopentyl, isomers of —O-hexyl, isomers of —O-cyclohexyl, etc.; C₁₋₄ hydroxyalkyl, such as —CH₂OH, —C₂H₄—OH, —C₃H₆—OH, C₄H₈—OH, etc.; C₂₋₅—CO₂-alkyl, such as —CO₂—CH₃, —CO₂—C₂H₅, —CO₂—C₃H₇, —CO₂—C₄H₉, etc.

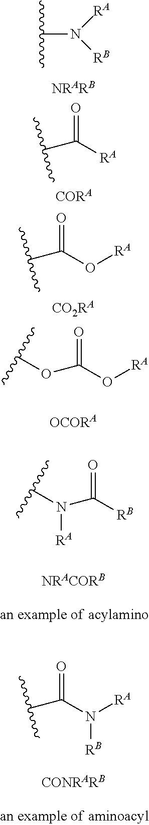

With respect to any relevant structural representation, each R^{A} may independently be H, or C₁₋₁₂ alkyl, including: linear or branched alkyl having a formula CaH₂ₐ₊₁, or cycloalkyl having a formula CaH₂ₐ₋₁, wherein a is 1, 2, 3, 4, 5, 6, 7, 8, 9, 10, 11, or 12, such as linear or branched alkyl of a formula: CH₃, C₂H₅, C₃H₇, C₄H₉, C₅H₁₁, C₆H₁₃, C₇H₁₅, C₈H₁₇, C₉H₁₉, C₁₀H₂₁, etc., or cycloalkyl of a formula: C₃H₅, C₄H₇, C₅H₉, C₆H₁₁, C₇H₁₃, C₈H₁₅, C₉H₁₇, C₁₀H₁₉, etc. In some embodiments, R^{A} may be H or C₁₋₆ alkyl. In some embodiments, R^{A} may be H or C₁₋₃ alkyl. In some embodiments, R^{A} may be H or CH₃. In some embodiments, R^{A} may be H.

With respect to any relevant structural representation, each R^{B} may independently be H, or C₁₋₁₂ alkyl, including: linear or branched alkyl having a formula CₐH₂ₐ₊₁, or cycloalkyl having a formula CₐH₂ₐ₋ₗ, wherein a is 1, 2, 3, 4, 5, 6, 7, 8, 9, 10, 11, or 12, such as linear or branched alkyl of a formula: CH₃, C₂H₅, C₃H₇, C₄H₉, C₅H₁₁, C₆H₁₃, C₇H₁₅, C₈H₁₇, C₉H₁₉, C₁₀H₂₁, etc., or cycloalkyl of a formula: C₃H₅, C₄H₇, C₅H₉, C₆H₁₁, C₇H₁₃, C₈H₁₅, C₉H₁₇, C₁₀H₁₉, etc. In some embodiments, R^{B} may be H or C₁₋₃ alkyl. In some embodiments, R^{B} may be H or CH₃. In some embodiments, R^{B} may be H.

With respect to any relevant structural representation, such as Formula 1, 1A, 1B, 1C, 2, 2A, 3, 3A, 4, 4A, 5, 5A, 6, 6A, 7, 7A, 8, or 9, R¹ is optionally substituted C₁₋₆ alkyl or optionally substituted C₃₋₁₀ cycloalkyl. If R¹ is substituted C₃₋₁₀ cycloalkyl, it may have 1, 2, 3, 4, 5, 6, 7, 8, 9, 10, or 11 substituents. R¹ may include any substituent. In some embodiments, some or all of the substituents of R¹ may have from 0 to 10 carbon atoms and from 0 to 10 heteroatoms, wherein each heteroatom is independently: O, N, S, F, Cl, Br, or I (provided that there is at least 1 non-hydrogen atom); and/or a molecular weight of 15 g/mol to 500 g/mol. In some embodiments, some or all of the substituents may each have a molecular weight of 15 Da to 200 Da, 15 Da to 100 Da, or 15 Da to 50 Da, and consist of 2 to 5 chemical elements, wherein the chemical elements are independently C, H, O, N, S, F, Cl, or Br. In some embodiments, R¹ is optionally substituted C₁₋₆ alkyl or optionally substituted C₃₋₁₀ cycloalkyl, such as CH₃, C₂H₅, C₃H₇, cyclic C₃H₅, C₄H₉, cyclic C₄H₇, C₅H₁₁, cyclic C₅H₉, C₆H₁₃, cyclic C₆H₁₁, cyclic C₇H₁₃, cyclic C₈H₁₅, cyclic C₉H₁₇, cyclic C₁₀H₁₉, etc.; C₁-10 optionally substituted alkoxy such as OCH₃, OC₂H₅, OC₃H₇, cyclic OC₃H₅, OC₄H₉, cyclic OC₄H₇, OC₅H₁₁, cyclic OC₅H₉, OC₆H₁₃, cyclic OC₆H₁₁, etc.; halo, such as F, Cl, Br, I; OH; CN; NO₂; C₁₋₆ fluoroalkyl, such as CF₃, CF₂H, C₂F₅, etc.; C₁₋₆ fluoroalkoxy, such as OCF₃, OCF₂H, OC₂F₅, etc.; a C₁₋₁₀ ester such as —O₂CCH₃, —CO₂CH₃, —O₂CC₂H₅, —CO₂C₂H₅, —O₂C-phenyl, —CO₂-phenyl, etc.; a C₁₋₁₀ ketone such as —COCH₃, —COC₂H₅, —COC₃H₇, —CO-phenyl, etc.; or a C₁₋₁₀ amine such as NH₂, NH(CH₃), N(CH₃)₂, N(CH₃)C₂H₅, etc. In some embodiments, a substituent of D may be F, Cl, Br, I, CN, NO₂, C₁₋₄ alkyl, C₁₋₄ alkyl-OH, C₁₋₃ O-alkyl, CF₃, C(O)H, C₁₋₄ CO-alkyl, CO₂H, C₁₋₄ CO₂-alkyl, NH₂, or C₁₋₄ alkylamino.

With respect to any relevant structural representation, such as Formula 1, 1A, 1B, 1C, 2, 2A, 3, 3A, 4, 4A, 5, 5A, 6, 6A, 7, 7A, 8, or 9, in some embodiments, R¹ is optionally substituted bicycloheptanyl or optionally substituted cyclopentanyl. In some embodiments, R¹ is optionally substituted bicycloheptanyl. In some embodiments, R¹ is optionally substituted bicyclo[2.2.1]heptanyl,

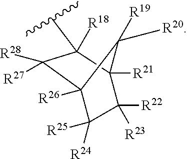

With respect to any relevant structural representation, such as Formula 1, 1A, 1B, 1C, 2, 2A, 3, 3A, 4, 4A, 5, 5A, 6, 6A, 7, 7A, 8, or 9, in some embodiments, R¹ is unsubstituted bicyclo[2.2.1]heptanyl,

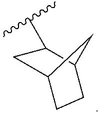 [SMILES: *C1CC2CCC1C2]

With respect to any relevant structural representation, such as Formula 1, 1A, 1B, 1C, 2, 2A, 3, 3A, 4, 4A, 5, 5A, 6, 6A, 7, 7A, 8, or 9, in some embodiments, R¹ is optionally substituted cyclopentanyl. In some embodiments, R¹ is unsubstituted cyclopentanyl,

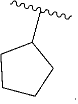 [SMILES: *C1CCCC1]

With respect to any relevant structural representation, such as Formula 1, 1A, 1B, 1C, 2, 2A, 3, 3A, 8, or 9, in some embodiments, R^{1a} is H, COR⁸, optionally substituted C₁₋₆ alkyl, or optionally substituted C₃₋₆ cycloalkyl. In some embodiments, R^{1a} is H, COCH₃, or CH₃. In some embodiments, R^{1a} is H or CH₃. In some embodiments, R^{1a} is H. In some embodiments, R^{1a} is COCH₃.

With respect to any relevant structural representation, such as Formula 1, 1A, 1B, 1C, 2, 2A, 3, 3A, 8, or 9, in some embodiments, R^{1b} is H, optionally substituted C₁₋₆ alkyl, or optionally substituted C₃₋₆ cycloalkyl. In some embodiments, R^{1b} is H, or CH₃. In some embodiments, R^{1b} H or CH₃. In some embodiments, R^{1b} is H. In some embodiments, R^{1b} CH₃.

With respect to any relevant structural representation, such as Formula 1, 1A, 1B, or 1C, in some embodiments, R^{1c} is H, CN, OH, optionally substituted hydrocarbyl, alkoxy, NR⁹R⁸, optionally substituted aryl or optionally substituted heteroaryl. In some embodiments, R^{1c} is H, OH, CH₃, OCH₃, or NH₂. In some embodiments, R^{1c} is H, OH or CH₃. In some embodiments, R^{1c} is H. In some embodiments, R^{1c} is OH. In some embodiments R^{1c} is CH₃.

With respect to any relevant structural representation, such as Formula 1, 5, 5A, 7, or 7A, in some embodiments, R¹¹ is R⁸, —OR⁸, SO₂R⁸, SO₂NR⁸R⁹, COR⁸, CO₂R⁸, or CONR⁸R⁹, wherein R⁸ and R⁹ are independently H, or C₁₋₆ hydrocarbyl optionally substituted with F, Cl, Br, I, amino, OH, C₁₋₆—O-alkyl, cyano, or a C₁₋₆ geminal -alkyl-O-alkyl-.

With respect to any relevant structural representation, such as Formula 1, 5, 5A, 7, or 7A, in some embodiments, R¹¹ is E-Hy.

With respect to E-Hy, E may be a bond; C₁₋₅ alkylene, such as C₁alkylene, C₂ alkylene, C₃ alkylene (including —(CH₂)₃—), C₄ alkylene (including —(CH₂)₂CH(CH₃)—), or C₅ alkylene; or C₁₋₅—O-alkylene, such as C₁—O-alkylene, C₂—O-alkylene, C₃—O-alkylene (including —O—(CH₂)CH(CH₃)—), C₄—O-alkylene, or C₅—O-alkylene. In some embodiments, E is —(CH₂)₃—. In some embodiments, E is —(CH₂)₂CH(CH₃)—. In some embodiments, E is C₁₋₅—O-alkylene-. In some embodiments, E is —O—(CH₂)CH(CH₃)—.

With respect to E-Hy, Hy may be OH or H. In some embodiments, Hy is OH. In some embodiments, Hy is H.

In some embodiments, R¹¹ is H, optionally substituted C₁₋₄ alkyl, or optionally substituted C₁₋₄ hydroxyalkyl. In some embodiments, R¹¹ is H. In some embodiments, R¹¹ is optionally substituted C₁₋₄ alkyl. In some embodiments, R¹¹ is optionally substituted C₁₋₄ hydroxyalkyl. In some embodiments, R¹¹ is

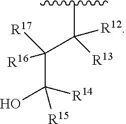

In some embodiments, R¹¹ is

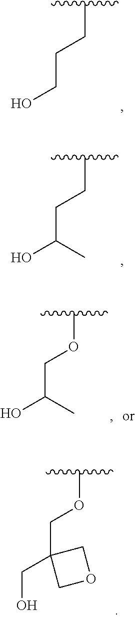

In some embodiments, R¹¹ is

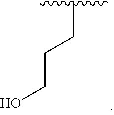 [SMILES: *CCCO]

In some embodiments, R¹¹ is

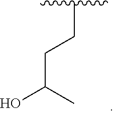 [SMILES: *CCC(C)O]

In some embodiments, R¹¹ is

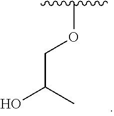 [SMILES: *OCC(C)O]

In some embodiments, R¹¹ is

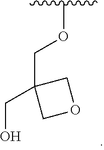 [SMILES: *OCC1(CO)COC1]

With respect to any relevant structural representation, such as Formula 1, 1A, 1B, 1C, 2, 2A, 3, 3A, 4, 4A, 5, 5A, 6, 6A, 7, 7A, 8, or 9, in some embodiments, R² is H, F, Cl, Br, I, cyano, OH, SOR⁸, SO₂R⁸, SO₂NR⁹R⁸, COR⁸, CO₂R⁸, CONR⁹R⁸, NR⁹R⁸, NR⁹COR⁸, NR⁹SO₂R⁸, NR⁹CO₂R⁸, NR⁹CONR⁸, OCOR⁸. In some embodiments, the R⁸ and R⁹ are free of heteratom-containing substituents. In some embodiments, R² is F or Cl. In some embodiments, R² is F.

In some embodiments, R^{2a} is F or H, and R^{2b}, R^{2c}, and R^{2d} are any of the groups recited above for R². In some embodiments, R^{2a} is F and R^{2b}, R^{2c}, and R^{2d} are any of the groups recited above for R². In some embodiments, R^{2a} is H, and R^{2b}, R^{2c}, and R^{2d} are any of the groups recited above for R².

In some embodiments, R^{2b} is F or H, and R^{2a}, R^{2c}, and R^{2d} are any of the groups recited above for R². In some embodiments, R^{2b} is F and R^{2a}, R^{2c}, and R^{2d} are any of the groups recited above for R². In some embodiments, R^{2b} is H, and R^{2a}, R^{2c}, and R^{2d} are any of the groups recited above for R².

With respect to any relevant structural representation, such as Formula 1, 1A, 1B, 1C, 2, 2A, 3, 3A, 4, 4A, 5, 5A, 6, 6A, 7, 7A, 8, or 9, in some embodiments, R³ is H, F, Cl, Br, I, cyano, OH, SOR⁸, SO₂R⁸, SO₂NR⁹R⁸, COR⁸, CO₂R⁸, CONR⁹R⁸, NR⁹R⁸, NR⁹COR⁸, NR⁹SO₂R⁸, NR⁹CO₂R⁸, NR⁹CONR⁸, OCOR⁸. In some embodiments, the R⁸ and R⁹ are free of heteroatom-containing substituents. In some embodiments, 1, 2, 3, or 4 R³ groups are H.

In some embodiments, R^{3a} is H, and R^{3b}, R^{3c}, and R^{3d} are any of the groups recited above for R³.

In some embodiments, R^{3b} is H, and R^{3a}, R^{3c}, and R^{3d} are any of the groups recited above for R³.

With respect to any relevant structural representation, such as Formula 1A, 1B, or 1C, in some embodiments, R⁴ is H or CH₃. In some embodiments, R⁴ is H. In some embodiments, R⁴ is CH₃.

With respect to any relevant structural representation, such as Z of Formula 1A or 1C, in some embodiments, R⁵ is R⁸, F, Cl, Br, I, cyano, —OR⁸, SOR⁸, SO₂R⁸, SO₂NR⁹R⁸, COR⁸, CO₂R⁸, CONR⁹R⁸, NR⁹R⁸, NR⁹COR⁸, NR⁹SO₂R⁸, NR⁹CO₂R⁸, NR⁹CONR⁸, or OCOR⁸.

With respect to any relevant structural representation, such as W of Formula 1A or 1C, in some embodiments, R⁶ is R⁸, F, Cl, Br, I, cyano, —OR⁸, SOR⁸, SO₂R⁸, SO₂NR⁹R⁸, COR⁸, CO₂R⁸, CONR⁹R⁸, NR⁹R⁸, NR⁹COR⁸, NR⁹SO₂R⁸, NR⁹CO₂R⁸, NR⁹CONR⁸, or OCOR⁸.

With respect to any relevant structural representation, in some embodiments, R⁷ is R⁸, F, Cl, Br, I, cyano, —OR⁸, SOR⁸, SO₂R⁸, SO₂NR⁹R⁸, COR⁸, CO₂R⁸, CONR⁹R⁸, NR⁹R⁸, NR⁹COR⁸, NR⁹SO₂R⁸, NR⁹CO₂R⁸, NR⁹CONR⁸, or OCOR⁸.

With respect to any relevant structural representation, such as Formula 1, 1A, 1B, 1C, 2, 2A, 3, 3A, 4, 4A, 5, 5A, 6, 6A, 7, 7A, 8, or 9, in some embodiments, R⁸ is H; or R⁸ is C₁₋₆ hydrocarbyl (such as C₁₋₆ alkyl, C₃₋₆ cycloalkyl, C₂₋₆ alkenyl, C₃₋₆ cycloalkenyl, C₂₋₆ alkynyl, or C₃₋₆ cycloalkenyl), which can be optionally substituted with F, Cl, Br, I, amino, hydroxyl, C₁₋₆ alkoxy or cyano.

With respect to any relevant structural representation, such as Formula 1, 1A, 1B, 1C, 2, 2A, 3, 3A, 4, 4A, 5, 5A, 6, 6A, 7, 7A, 8, or 9, in some embodiments, R⁹ is H, or R⁹ is C₁₋₆ hydrocarbyl (such as C₁₋₆ alkyl, C₃₋₆ cycloalkyl, C₂₋₆ alkenyl, C₃₋₆ cycloalkenyl, C₂₋₆ alkynyl, or C₃₋₆ cycloalkenyl), which can be optionally substituted with F, Cl, Br, I, amino, hydroxyl, C₁₋₆ alkoxy or cyano.

With respect to any relevant structural representation, in some embodiments, R¹² is H, and the remaining groups of R¹⁻²⁸ are any of the relevant groups recited above. In some embodiments, R¹³ is H, and the remaining groups of R¹⁻²⁸ are any of the relevant groups recited above. In some embodiments, R¹⁴ is H, and the remaining groups of R¹⁻²⁸ are any of the relevant groups recited above. In some embodiments, R¹⁵ is H, and the remaining groups of R¹⁻²⁸ are any of the relevant groups recited above. In some embodiments, R¹⁶ is H, and the remaining groups of R¹⁻²⁸ are any of the relevant groups recited above. In some embodiments, R¹⁷ is H, and the remaining groups of R¹⁻²⁸ are any of the relevant groups recited above. In some embodiments, R¹⁸ is H, and the remaining groups of R¹⁻²⁸ are any of the relevant groups recited above. In some embodiments, R¹⁹ is H, and the remaining groups of R¹⁻²⁸ are any of the relevant groups recited above. In some embodiments, R²⁰ is H, and the remaining groups of R¹⁻²⁸ are any of the relevant groups recited above. In some embodiments, R²¹ is H, and the remaining groups of R¹⁻²⁸ are any of the relevant groups recited above. In some embodiments, R²² is H, and the remaining groups of R¹⁻²⁸ are any of the relevant groups recited above. In some embodiments, R²³ is H, and the remaining groups of R¹⁻²⁸ are any of the relevant groups recited above. In some embodiments, R²⁴ is H, and the remaining groups of R¹⁻²⁸ are any of the relevant groups recited above. In some embodiments, R²⁵ is H, and the remaining groups of R¹⁻²⁸ are any of the relevant groups recited above. In some embodiments, R²⁶ is H, and the remaining groups of R¹⁻²⁸ are any of the relevant groups recited above. In some embodiments, R²⁷ is H, and the remaining groups of R¹⁻²⁸ are any of the relevant groups recited above. In some embodiments, R²⁸ is H, and the remaining groups of R¹⁻²⁸ are any of the relevant groups recited above.

With respect to any relevant structural representation, such as Formula 2A, 3A, 4A, 6A, 7A, 8, or 9, X is CH or N. In some embodiments, X is CH. In some embodiments, X is N.

With respect to any relevant structural representation, such as Formula 1A, 1C, 2, 2A, 4, 4A, 6, 6A, or 8, n is 1, 2, or 3. In some embodiments, n is 1. In some embodiments, n is 2. In some embodiments, n is 3.

For some compounds represented by Formula 1A, 1B or 1C:

R¹ is optionally substituted C₁₋₆ alkyl or optionally substituted C₃₋₁₀ cycloalkyl; A is optionally 1-4 R², (same or different) substituted aryl or optionally 1-3 R² (same or different) substituted heteroaryl; R^{1a} is H or COCH₃; R^{1b} is H or CH₃; R^{1c} is H, OH or optionally substituted hydrocarbyl; Z is C(R⁵)₂; W is C(R⁶)₂; m=1 or 2; n=0, 1 or 2; and R⁴ is hydrogen or CH₃;

each R^{3a}, R^{3b}, R^{3c}, R^{3d} of Formulas 1, 1A, 1B or 1C is independently H or C₁₋₆ hydrocarbyl, optionally substituted with one or two, and same or different R⁷; and R⁴ is H or CH₃;

each R², R⁵, R⁶, R⁷ of Formulas 1, 1A, 1B or 1C is independently selected from R⁸, F, Cl, Br, I, cyano, —OR⁸, C₁₋₆ hydrocarbyl, C₁₋₆ alkoxy, SOR⁸, SO₂R⁸, SO₂NR⁹R⁸, COR⁸, CO₂R⁸, CONR⁹R⁸, NR⁹R⁸, NR⁹COR⁸, NR⁹SO₂R⁸, NR⁹CO₂R⁸, NR⁹CONR⁸, OCOR⁸, wherein each of C₁₋₆ hydrocarbyl, C₁₋₆ alkoxy, SOR⁸, SO₂R⁸, SO₂NR⁹R⁸, COR⁸, CO₂R⁸, CONR⁹R⁸, NR⁹R⁸, NR⁹COR⁸, NR⁹SO₂R⁸, NR⁹CO₂R⁸, NR⁹CONR⁸, or OCOR⁸.

R⁸ and R⁹ are independently H, or C₁₋₆ hydrocarbyl, wherein each of the C₁₋₆ hydrocarbyl can be optionally substituted with F, Cl, Br, I, amino, hydroxyl, C₁₋₆ alkoxy or cyano.

With respect to any relevant structural representation, such as Formula 2 or Formula 2A, in some embodiments R^{1a} is H or COCH₃; R^{1b} is H or CH₃; and n is 1 or 2.

With respect to any relevant structural representation, such as Formula 3, Formula 3A, or Formula 9, R^{1a} is H or COCH₃; and R^{1b} is H or CH₃.

With respect to any relevant structural representation, such as Formula 6A, in some embodiments, X is CH or N; and when X is CH, at least one R² is not H.

With respect to any relevant structural representation, such as Formula 7A, X is CH or N; and when X is CH, R² is H.

With respect to any relevant structural representation, such as Formula 7 or Formula 7A, R¹ is optionally substituted bicycloheptanyl.

With respect to any relevant structural representation, such as Formula 8, X is CH or N; R^{1a} is H or COCH₃; R^{1b} is H or CH₃; and n is 1 or 2.

With respect to any relevant structural representation, such as Formula 1B, in some embodiments, m is 1 or 2. In some embodiments, m is 1. In some embodiments, m is 2.

With respect to any relevant structural representation, such as Formula 1A, 1C, 2, 2A, 4, 4A, 6, 6A or 8, n may be 1 or 2. In some embodiments, n may be 0, such as compound represented by Formula 1A or 1C. In some embodiments, n may be 3, such as compounds represented by Formula 4 or 4A.

Some embodiments include one or more of the following:

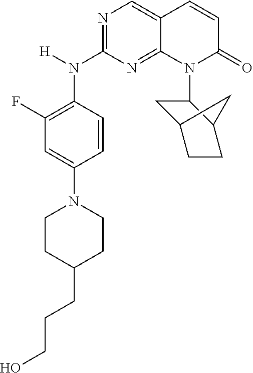 [SMILES: O=c1ccc2cnc(Nc3ccc(N4CCC(CCCO)CC4)cc3F)nc2n1C1CC2CCC1C2]

### 8-((2R)-bicyclo[2.2.1]heptan-2-yl)-2-((2-fluoro-4-(4-(3-hydroxypropyl)piperidin-1-yl)phenyl)amino)pyrido[2,3-d]pyrimidin-7(8H)-one (PS002)

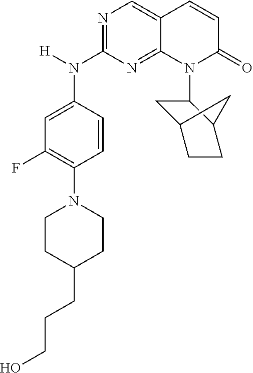 [SMILES: O=c1ccc2cnc(Nc3ccc(N4CCC(CCCO)CC4)c(F)c3)nc2n1C1CC2CCC1C2]

### 8-((2R)-bicyclo[2.2.1]heptan-2-yl)-2-((3-fluoro-4-(4-(3-hydroxypropyl)piperidin-1-yl)phenyl)amino)pyrido[2,3-d]pyrimidin-7(8H)-one (PS003)

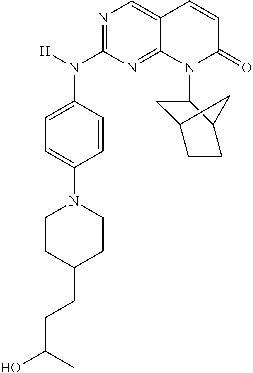 [SMILES: CC(O)CCC1CCN(c2ccc(Nc3ncc4ccc(=O)n(C5CC6CCC5C6)c4n3)cc2)CC1]

### 8-((2R)-bicyclo[2.2.1]heptan-2-yl)-2-((4-(4-(3-hydroxybutyl)piperidin-1-yl)phenyl)amino)pyrido[2,3-d]pyrimidin-7(8H)-one (PS004)

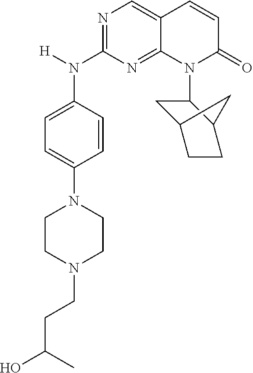 [SMILES: CC(O)CCN1CCN(c2ccc(Nc3ncc4ccc(=O)n(C5CC6CCC5C6)c4n3)cc2)CC1]

### 8-((2R)-bicyclo[2.2.1]heptan-2-yl)-2-((4-(4-(3-hydroxybutyl)piperazin-1-yl)phenyl)amino)pyrido[2,3-d]pyrimidin-7(8H)-one (PS005)

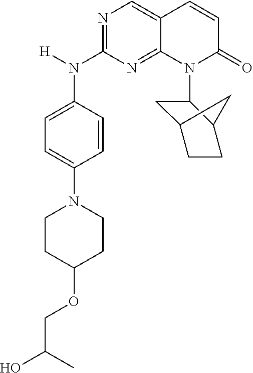 [SMILES: CC(O)COC1CCN(c2ccc(Nc3ncc4ccc(=O)n(C5CC6CCC5C6)c4n3)cc2)CC1]

### 8-((2R)-bicyclo[2.2.1]heptan-2-yl)-2-((4-(4-(2-hydroxypropoxy)piperidin-1-yl)phenyl)amino)pyrido[2,3-d]pyrimidin-7(8H)-one (PS006)

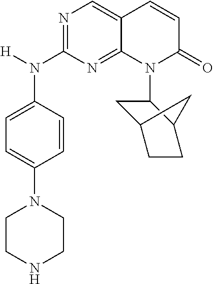 [SMILES: O=c1ccc2cnc(Nc3ccc(N4CCNCC4)cc3)nc2n1C1CC2CCC1C2]

### 8-((2R)-bicyclo[2.2.1]heptan-2-yl)-2-((4-(piperazin-1-yl)phenyl)amino)pyrido[2,3-d]pyrimidin-7(8H)-one (PS007)

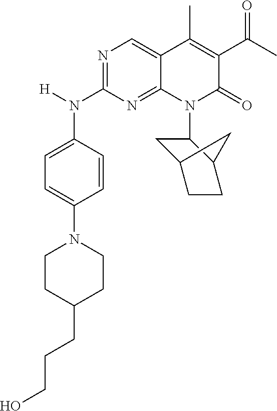 [SMILES: CC(=O)c1c(C)c2cnc(Nc3ccc(N4CCC(CCCO)CC4)cc3)nc2n(C2CC3CCC2C3)c1=O]

### 6-acetyl-8-((2R)-bicyclo[2.2.1]heptan-2-yl)-2-((4-(4-(3-hydroxypropyl)piperidin-1-yl)phenyl)amino)-5-methylpyrido[2,3-d]pyrimidin-7(8H)-one (PS008)

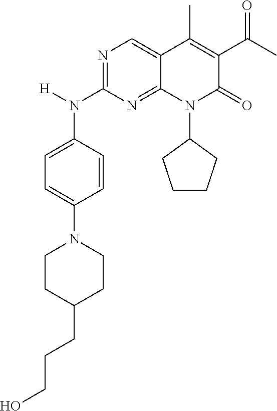 [SMILES: CC(=O)c1c(C)c2cnc(Nc3ccc(N4CCC(CCCO)CC4)cc3)nc2n(C2CCCC2)c1=O]

### 6-acetyl-8-cyclopentyl-2-((4-(4-(3-hydroxypropyl)piperidin-1-yl)phenyl)amino)-5-methylpyrido[2,3-d]pyrimidin-7(8H)-one (PS009)

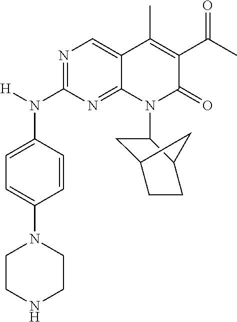 [SMILES: CC(=O)c1c(C)c2cnc(Nc3ccc(N4CCNCC4)cc3)nc2n(C2CC3CCC2C3)c1=O]

### 6-acetyl-8-((2R)-bicyclo[2.2.1]heptan-2-yl)-5-methyl-2-((4-(piperazin-1-yl)phenyl)amino)pyrido[2,3-d]pyrimidin-7(8H)-one (PS010)

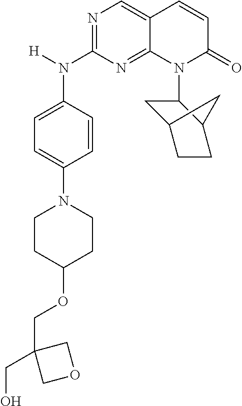 [SMILES: O=c1ccc2cnc(Nc3ccc(N4CCC(OCC5(CO)COC5)CC4)cc3)nc2n1C1CC2CCC1C2]

### 8-((2R)-bicyclo[2.2.1]heptan-2-yl)-2-((4-(4-((3-(hydroxymethyl)oxetan-3-yl)methoxy)piperidin-1-yl)phenyl)amino)pyrido[2,3-d]pyrimidin-7(8H)-one (PS016)

Some embodiments include optionally substituted 8-((2R)-bicyclo[2.2.1]heptan-2-yl)-2-((2-fluoro-4-(4-(3-hydroxypropyl)piperidin-1-yl)phenyl)amino)pyrido[2,3-d]pyrimidin-7(8H)-one; optionally substituted 8-((2R)-bicyclo[2.2.1]heptan-2-yl)-2-((3-fluoro-4-(4-(3-hydroxypropyl)piperidin-1-yl)phenyl)amino)pyrido[2,3-d]pyrimidin-7(8H)-one; optionally substituted 8-((2R)-bicyclo[2.2.1]heptan-2-yl)-2-((4-(4-(3-hydroxybutyl)piperidin-1-yl)phenyl)amino)pyrido[2,3-d]pyrimidin-7(8H)-one; optionally substituted 8-((2R)-bicyclo[2.2.1]heptan-2-yl)-2-((4-(4-(3-hydroxybutyl)piperazin-1-yl)phenyl)amino)pyrido[2,3-d]pyrimidin-7(8H)-one; optionally substituted 8-((2R)-bicyclo[2.2.1]heptan-2-yl)-2-((4-(4-(2-hydroxypropoxy)piperidin-1-yl)phenyl)amino)pyrido[2,3-d]pyrimidin-7(8H)-one; optionally substituted 8-((2R)-bicyclo[2.2.1]heptan-2-yl)-2-((4-(piperazin-1-yl)phenyl)amino)pyrido[2,3-d]pyrimidin-7(8H)-one; optionally substituted 6-acetyl-8-((2R)-bicyclo[2.2.1]heptan-2-yl)-2-((4-(4-(3-hydroxypropyl)piperidin-1-yl)phenyl)amino)-5-methylpyrido[2,3-d]pyrimidin-7(8H)-one; optionally substituted 6-acetyl-8-cyclopentyl-2-((4-(4-(3-hydroxypropyl)piperidin-1-yl)phenyl)amino)-5-methylpyrido[2,3-d]pyrimidin-7(8H)-one; or optionally substituted 6-acetyl-8-((2R)-bicyclo[2.2.1]heptan-2-yl)-5-methyl-2-((4-(piperazin-1-yl)phenyl)amino)pyrido[2,3-d]pyrimidin-7(8H)-one; or optionally substituted 8-((2R)-bicyclo[2.2.1]heptan-2-yl)-2-((4-(4-((3-(hydroxymethyl)oxetan-3-yl)methoxy)piperidin-1-yl)phenyl)amino)pyrido[2,3-d]pyrimidin-7(8H)-one.

A subject compound can be used to treat a disorder or disease associated with a CDK inhibitor. Treatment of a disorder includes diagnosis, cure, mitigation, treatment, or prevention of the disorder in man or animals. In some embodiments, the disease is cancer. In some embodiments, the disease or disorder may include breast cancer, melanoma, renal cancer, squamous cell carcinoma, bladder cancer, pancreatic cancer, ovarian cancer, lung cancer, prostate cancer, colon cancer, esophageal cancer, head cancer, neck cancer, neuroblastoma, myeloma, glioma, lymphomas and leukemias.

Appropriate excipients for use in a subject composition may include, for example, one or more carriers, binders, fillers, vehicles, disintegrants, surfactants, dispersion or suspension aids, thickening or emulsifying agents, isotonic agents, preservatives, lubricants, and the like or combinations thereof, as suited to a particular dosage from desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof.

A subject composition may be formulated for any desirable route of delivery including, but not limited to, parenteral, intravenous, intradermal, subcutaneous, oral, inhalative, transdermal, topical, transmucosal, rectal, interacisternal, intravaginal, intraperitoneal, buccal, and intraocular.

Parenteral, intradermal or subcutaneous formulations may be sterile injectable aqueous or oleaginous suspensions or solutions. Acceptable vehicles, solutions, suspensions and solvents may include, but are not limited to, water or other sterile diluent; saline; Ringer's solution; sodium chloride; fixed oils such as mono- or diglycerides; fatty acids such as oleic acid; polyethylene glycols; glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates; and agents for the adjustment of tonicity such as sodium chloride or dextrose. The pH can be adjusted with acids or bases, such as hydrochloric acid or sodium hydroxide. A parenteral preparation may be enclosed in ampoules, disposable syringes or multiple dose vials made of glass or plastic.

Pharmaceutical compositions suitable for injectable use may include sterile aqueous solutions or dispersions and sterile powders for the extemporaneous preparation of sterile injectable solutions or dispersion. For intravenous administration, suitable carriers include, but are not limited to, saline, bacteriostatic water, CREMOPHOR EL® (BASF, Parsippany, N.J.) or phosphate buffered saline (PBS). The solvent or dispersion medium may contain, for example, water, ethanol, polyol (for example, glycerol, propylene glycol, and liquid polyethylene glycol, and the like), and suitable mixtures thereof. Proper fluidity can be maintained, for example, by the use of a coating such as lecithin, by the maintenance of the required particle size in the case of dispersion and by the use of surfactants. Preventing growth of microorganisms can be achieved by various antibacterial and antifungal agents, for example, parabens, chlorobutanol, phenol, ascorbic acid, thimerosal, and the like. The composition may also include isotonic agents such as, for example, sugars; polyalcohols such as mannitol; sorbitol; or sodium chloride.

Prolonged absorption of injectable compositions can be enhanced by addition of an agent that delays absorption, such as, for example, aluminum monostearate or gelatin.

Oral compositions may include an inert diluent or an edible carrier. They may be enclosed in gelatin capsules or compressed into tablets. Tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

In addition to oral or injected administration, systemic administration may be by transmucosal or transdermal means. For transmucosal or transdermal administration, penetrants may be used. Such penetrants are generally known in the art and include, for example, detergents, bile salts, and fusidic acid derivatives. Transdermal administration may include a bioactive agent and may be formulated into ointments, salves, gels, or creams as generally known in the art. Transmucosal administration may be accomplished through the use of nasal sprays or suppositories.

A subject compound may be administered in a therapeutically effective amount, according to an appropriate dosing regimen. As understood by a skilled artisan, an exact amount required may vary from subject to subject, depending on a subject's species, age and general condition, the severity of the infection, the particular agent(s) and the mode of administration. In some embodiments, about 0.001 mg/kg to about 50 mg/kg, of the pharmaceutical composition based on the subject's body weight is administered, one or more times a day, to obtain the desired therapeutic effect. In other embodiments, about 0.01 mg/kg to about 25 mg/kg, of the pharmaceutical composition based on the subject's body weight is administered, one or more times a day, to obtain the desired therapeutic effect.

A total daily dosage of a subject compound can be determined by the attending physician within the scope of sound medical judgment. A specific therapeutically effective dose level for any particular patient or subject will depend upon a variety of factors including the disorder being treated and the severity of the disorder; the activity of the specific compound employed; the specific composition employed; the age, body weight, general health, sex and diet of the patient or subject; the time of administration, route of administration, and rate of excretion of the specific compound employed; the duration of the treatment; drugs used in combination or coincidental with the specific compound employed; and other factors well known in the medical arts.

The following embodiments are specifically contemplated herein.

Embodiment 1. A compound represented by a formula:

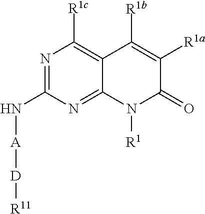

or a salt thereof;

wherein R¹ is optionally substituted C₁₋₆ alkyl or optionally substituted C₃₋₁₀ cycloalkyl;

R^{1a} is H or COCH₃;

R^{1b} is H or CH₃;

R^{1c} is H;

A is optionally substituted aryl or optionally substituted heteroaryl;

D is optionally substituted piperidin-1,4-yl or optionally substituted piperazin-1,4-yl;

R¹¹ is R⁸, —OR⁸, SO₂R⁸, SO₂NR⁸R⁹, COR⁸, CO₂R⁸, or CONR⁸R⁹, wherein R⁸ and R⁹ are independently H, or C₁₋₆ hydrocarbyl optionally substituted with F, Cl, Br, I, amino, OH, C₁₋₆—O-alkyl, cyano, or a C₁₋₆ geminal -alkyl-O-alkyl-.

Embodiment 2. The compound of embodiment 1, further represented by a formula:

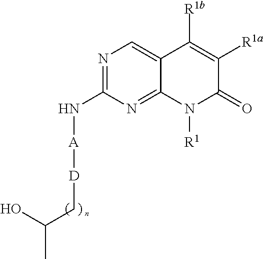

or a salt thereof;

wherein A is optionally substituted p-phenylene, or optionally substituted pyridin-2,5-yl; wherein the 2-position attaches to NH and the 5-position attaches to D;

D is optionally substituted piperidin-1,4-yl, wherein the 1-position attaches to A;

R^{1a} is H or COCH₃;

R^{1b} is H or CH₃; and

n is 1 or 2.

Embodiment 3. The compound of embodiment 1, further represented by a formula:

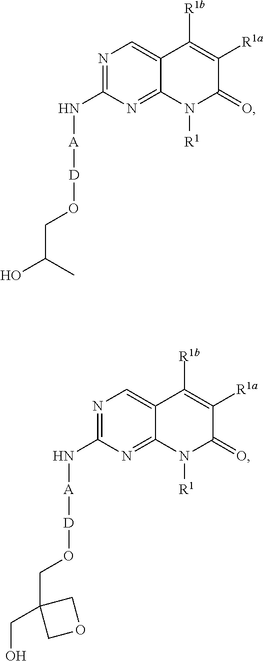

or a salt thereof;

wherein A is optionally substituted p-phenylene, or optionally substituted pyridin-2,5-yl wherein the 2-position attaches to NH and the 5-position attaches to D;

D is optionally substituted piperidin-1,4-yl wherein the 1-position attaches to A;

R^{1a} is H or COCH₃; and

R^{1b} is H or CH₃.

Embodiment 4. The compound of embodiment 1, further represented by a formula:

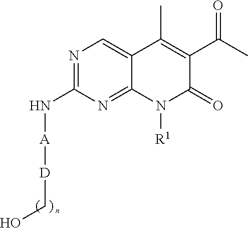

or a salt thereof;

wherein A is optionally substituted p-phenylene, or optionally substituted pyridin-2,5-yl wherein the 2-position attaches to NH and the 5-position attaches to D;

D is optionally substituted piperidin-1,4-yl wherein the 1-position attaches to A; and

n is 1, 2, or 3.

Embodiment 5. The compound of embodiment 1, further represented by a formula:

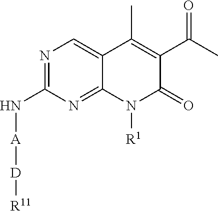

or a salt thereof;

wherein R¹ is optionally substituted bicycloheptanyl;

A is optionally substituted p-phenylene; and

D is unsubstituted piperazin-1,4-yl.

Embodiment 6. The compound of embodiment 1, further represented by a formula:

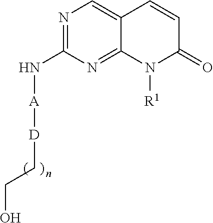

wherein A is substituted p-phenylene, or optionally substituted pyridin-2,5-yl wherein the 2-position attaches to NH and the 5-position attaches to D;

D is optionally substituted piperidin-1,4-yl wherein the 1-position attaches to A; and

n is 1 or 2.

Embodiment 7. The compound of embodiment 1, further represented by a formula:

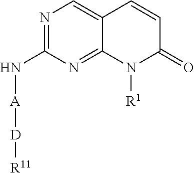

or a salt thereof;

wherein R¹ is optionally substituted bicycloheptanyl; and

A is optionally substituted pyridin-2,5-yl wherein the 2-position attaches to NH and the 5-position attaches to D, and D is optionally substituted piperazin-1,4-yl; or A is optionally substituted phenyl and D is unsubstituted piperazin-1,4-yl.

Embodiment 8. The compound of embodiment 1, 2, 3, 4, 5, 6, or 7, wherein R¹ is optionally substituted cyclopentanyl.

Embodiment 9. The compound of embodiment 8, wherein R¹ is unsubstituted cyclopentanyl.

Embodiment 10. The compound of embodiment 1, 2, 3, 4, 5, 6, or 7, wherein R¹ is optionally substituted bicyclo[2.2.1]heptanyl.

Embodiment 11. The compound of embodiment 10, wherein R¹ is unsubstituted bicyclo[2.2.1]heptanyl.

Embodiment 12. The compound of embodiment 1, 2, 3, 8, 9, 10, or 11, wherein R^{1a} is H.

Embodiment 13. The compound of embodiment 1, 2, 3, 8, 9, 10, or 11, wherein R^{1a} is COCH₃.

Embodiment 14. The compound of embodiment 1, 2, 3, 8, 9, 10, 11, 12, or 13, wherein R^{1b} is H.

Embodiment 15. The compound of embodiment 1, 2, 3, 8, 9, 10, 11, 12, or 13, wherein R^{1b} CH₃.

Embodiment 16. The compound of embodiment 1, 2, 3, 4, 5, 6, 7, 8, 9, 10, 11, 12, 13, 14, or 15, wherein A is optionally substituted p-phenylene.

Embodiment 17. The compound of embodiment 1, 2, 3, 4, 5, 6, 7, 8, 9, 10, 11, 12, 13, 14, or 15, wherein A is unsubstituted p-phenylene.

Embodiment 18. The compound of embodiment 1, 2, 3, 4, 5, 6, 7, 8, 9, 10, 11, 12, 13, 14, or 15, wherein A is fluoro-p-phenylene.

Embodiment 19. The compound of embodiment 1, 2, 3, 4, 5, 6, 7, 8, 9, 10, 11, 12, 13, 14, 15, 16, 17, or 18, wherein D is optionally substituted piperidin-1,4-yl.

Embodiment 20. The compound of embodiment 1, 2, 3, 4, 5, 6, 7, 8, 9, 10, 11, 12, 13, 14, 15, 16, 17, or 18, wherein D is unsubstituted piperidin-1,4-yl.

Embodiment 21. The compound of embodiment 1, 2, 3, 4, 5, 6, 7, 8, 9, 10, 11, 12, 13, 14, 15, 16, 17, or 18, wherein D is optionally substituted piperazin-1,4-yl.

Embodiment 22. The compound of embodiment wherein 1, 2, 3, 4, 5, 6, 7, 8, 9, 10, 11, 12, 13, 14, 15, 16, 17, or 18, wherein D is unsubstituted piperazin-1,4-yl.

Embodiment 23. The compound of embodiment 1, 2, 3, 4, 5, 6, 7, 8, 9, 10, 11, 12, 13, 14, 15, 16, 17, 18, 19, 20, 21, or 22, wherein R¹¹ is E-Hy, wherein E is a bond, C₁₋₅ alkylene, C₁₋₅ O-alkylene, or

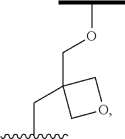

### and Hy is OH or H.

Embodiment 24. The compound of embodiment 23, wherein E is optionally substituted C₁₋₅ alkylene.

Embodiment 25. The compound of embodiment 23, wherein E is —(CH₂)₃—.

Embodiment 26. The compound of embodiment 23, wherein E is —(CH₂)₂CH(CH₃)—.

Embodiment 27. The compound of embodiment 23, wherein E is C₁₋₅—O-alkylene-.

Embodiment 28. The compound of embodiment 23 wherein E is —O—(CH₂)CH(CH₃)—.

Embodiment 29. The compound of embodiment 23 wherein E is

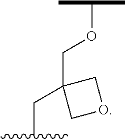

Embodiment 30. The compound of embodiment 23, 24, 25, 26, 27, 28, or 29, wherein Hy is OH.

Embodiment 31. The compound of embodiment 23, 24, 25, 26, 27, 28, or 29, wherein Hy is H.

Embodiment 32. A compound selected from:

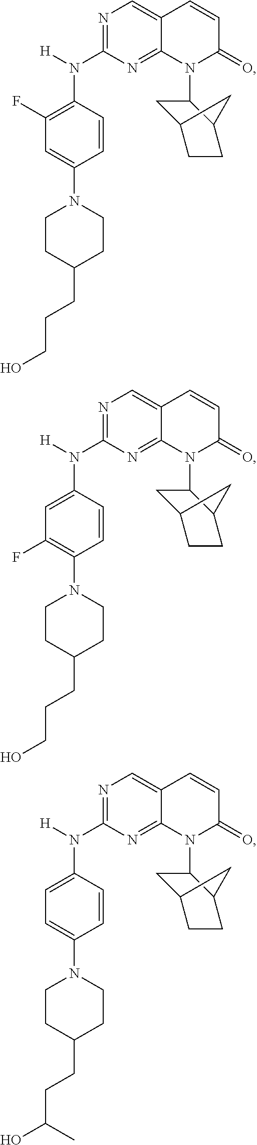 [SMILES: O=c1ccc2cnc(Nc3ccc(N4CCC(CCCO)CC4)cc3F)nc2n1C1CC2CCC1C2 ; O=c1ccc2cnc(Nc3ccc(N4CCC(CCCO)CC4)c(F)c3)nc2n1C1CC2CCC1C2 ; CC(O)CCC1CCN(c2ccc(Nc3ncc4ccc(=O)n(C5CC6CCC5C6)c4n3)cc2)CC1]

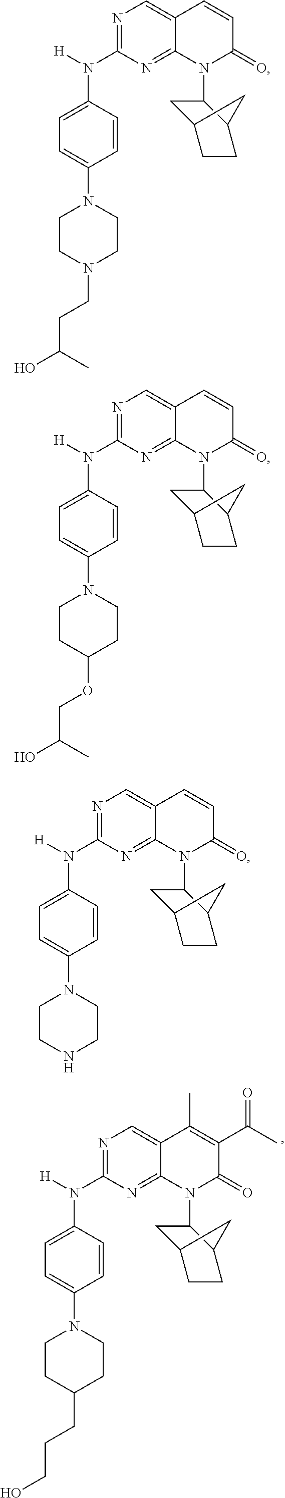 [SMILES: CC(O)CCN1CCN(c2ccc(Nc3ncc4ccc(=O)n(C5CC6CCC5C6)c4n3)cc2)CC1 ; CC(O)COC1CCN(c2ccc(Nc3ncc4ccc(=O)n(C5CC6CCC5C6)c4n3)cc2)CC1 ; O=c1ccc2cnc(Nc3ccc(N4CCNCC4)cc3)nc2n1C1CC2CCC1C2 ; CC(=O)c1c(C)c2cnc(Nc3ccc(N4CCC(CCCO)CC4)cc3)nc2n(C2CC3CCC2C3)c1=O]

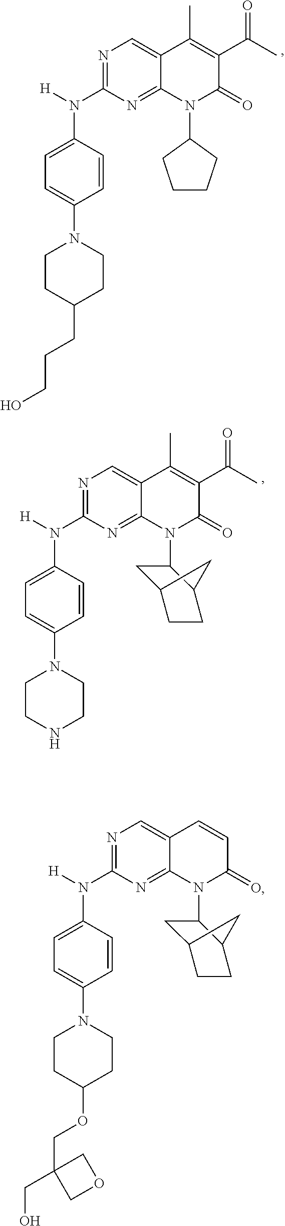 [SMILES: CC(=O)c1c(C)c2cnc(Nc3ccc(N4CCC(CCCO)CC4)cc3)nc2n(C2CCCC2)c1=O ; CC(=O)c1c(C)c2cnc(Nc3ccc(N4CCNCC4)cc3)nc2n(C2CC3CCC2C3)c1=O ; O=c1ccc2cnc(Nc3ccc(N4CCC(OCC5(CO)COC5)CC4)cc3)nc2n1C1CC2CCC1C2]

or a salt thereof.

Embodiment 33. A pharmaceutically acceptable composition comprising a compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32.

Embodiment 34. A pharmaceutical dosage form comprising a compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32.

Embodiment 35. A method of treating a disorder associated with a CDK inhibitor comprising administering an effective amount of a compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32.

Embodiment 36. The method of embodiment 35, wherein the disorder is cancer.

Embodiment 37. Use of a compound according to embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 in the manufacture of a medicament for the treatment of cancer.

Embodiment 38. The method or use of embodiment 36 or 37, wherein the cancer comprises breast cancer, renal cancer, bladder cancer, pancreatic cancer, ovarian cancer, lung cancer, prostate cancer, colon cancer, oesophageal cancer, head cancer, neck cancer, or leukemia.

Embodiment 39. The method of embodiment 35 further comprising administering an additional therapeutic agent.

Embodiment 40. The embodiment 39 wherein the additional therapeutic agent is an antibiotic, an antiemetic agent, an antidepressant, and antifungal agent, an anti-inflammatory agent, an antiviral agent, an anticancer agent, an immunomodulatory agent, an alkylating agent, or a hormone.

### EXPERIMENTAL SECTION

### Reference Materials:

Control commercial CDK inhibitors were obtained from Selleckchem: Palbociclib (PD-0332991)-HCl (#S1116), Abemaciclib (LY2835219) (#S7158) and Ribociclib (LEE011) (#S7440). PD-0183812, RO-3306 (#217699) and Roscovitine (#R7772) were obtained from WuXi AppTec, Merck Millipore and Sigma Aldrich, respectively. All compounds were reconstituted in DMSO (Sigma Aldrich) at 5 mM concentration (stock solution).

### Preparation of Compounds:

In the synthetic schemes described below, unless otherwise indicated all temperatures are set forth in degrees Celsius and all parts and percentages are by weight. Reagents and solvents were purchased from commercial suppliers such as Aldrich Chemical Company and were used without further purification unless otherwise indicated. Tetrahydrofuran (THF) and N,N-dimethylformamide (DMF) were purchased from Aldrich in Sure Seal bottles and used as received.

The reactions set forth below were done generally under a positive pressure of argon or nitrogen at an ambient temperature (unless otherwise stated) in anhydrous solvents. Glassware was oven dried and/or heat dried. The reactions were assayed by TLC and/or analyzed by LC-MS and terminated as judged by the consumption of starting material. Analytical thin layer chromatography (TLC) was performed on glass plates pre-coated with silica gel 60 F254 0.25 mm plates (EM Science), and visualized with UV light (254 nm) and/or heating with commercial ethanolic phosphomolybdic acid. preparative thin layer chromatography (TLC) was performed on glass-plates pre-coated with silica gel 60 F254 0.5 mm plates (20×20 cm, from Thomson Instrument Company) and visualized with UV light (254 nm).

Work-ups were typically done by doubling the reaction volume with the reaction solvent or extraction solvent and then washing with the indicated aqueous solutions using 25% by volume of the extraction volume unless otherwise indicated. Product solutions were dried over anhydrous Na₂SO₄ and/or Mg₂SO₄ prior to filtration and evaporation of the solvents under reduced pressure on a rotary evaporator and noted as solvents removed in vacuo. Column chromatography was completed under positive pressure using 230-400 mesh silica gel.

¹H-NMR spectra and ¹³C-NMR were recorded on a Varian Mercury-VX400 instrument operating at 400 MHZ. NMR spectra were obtained as CDCl₃ solutions (reported in ppm), using chloroform as the reference standard (7.27 ppm for the proton and 77.00 ppm for carbon), CD₃OD (3.4 and 4.8 ppm for the protons and 49.3 ppm for carbon), DMSO-d₆ (2.49 ppm for proton), or internally tetramethylsilane (0.00 ppm) when appropriate. Other NMR solvents were used as needed.

The compounds of the disclosure can be made using procedures known in the art. The following reaction schemes show typical procedures, but those skilled in the art will recognize that other procedures can also be suitable for using to prepare these compounds. For examples those skilled in the art will recognize that changes to the requisite reagents can be made at the appropriate steps in the synthetic methods outlined below. Reactions may involve monitoring for consumption of starting materials, and there are many methods for the monitoring, including but not limited to thin layer chromatography (TLC) and liquid chromatography mass spectrometry (LCMS). Those skilled in the art will recognize that any synthetic method specified in the examples shown below can be substituted by other non-limiting methods when suitable.

In some embodiments, the pyrido[2,3-d]pyrimidin-7-one analogues described herein are prepared by the general routes illustrated in Schemes 1 and 2. As shown in Scheme 1, condensation of commercially available 4-chloro-2-methylthio-5-pyrimidinecarboxylic acid ethyl ester (1) with a primary amine in THF containing triethylamine provides intermediates with structure 2. Reduction of the ester 2 using lithium aluminum hydride to alcohol 3, followed by re-oxidation with MnO₂, provides aldehyde 4. Reaction of the aldehyde 4 with (carbethoxymethylene)triphenylphosphorane in THF under reflux gives the undesired (E)-acrylate 5, which can be conveniently isomerized with ring closure to provide the pyrido[2,3-d]pyrimidine core molecule 6 by heating in the presence of DBU. Oxidation of the methyl sulfide in compound 6 with (+/−)-trans-2-(phenylsulfonyl)-3-phenyloxaziridine or m-chloroperbenzoic acid provides the corresponding sulfoxide 7 or sulfone 8. Introduction of amines at the C₂-position can be achieved by heating the sulfoxide or sulfone with no less than 2 equivalents of an amine such as an aromatic amine, in the presence or absence of a solvent, at temperatures ranging from 100° C. to 175° C., to provide the general structure 9.

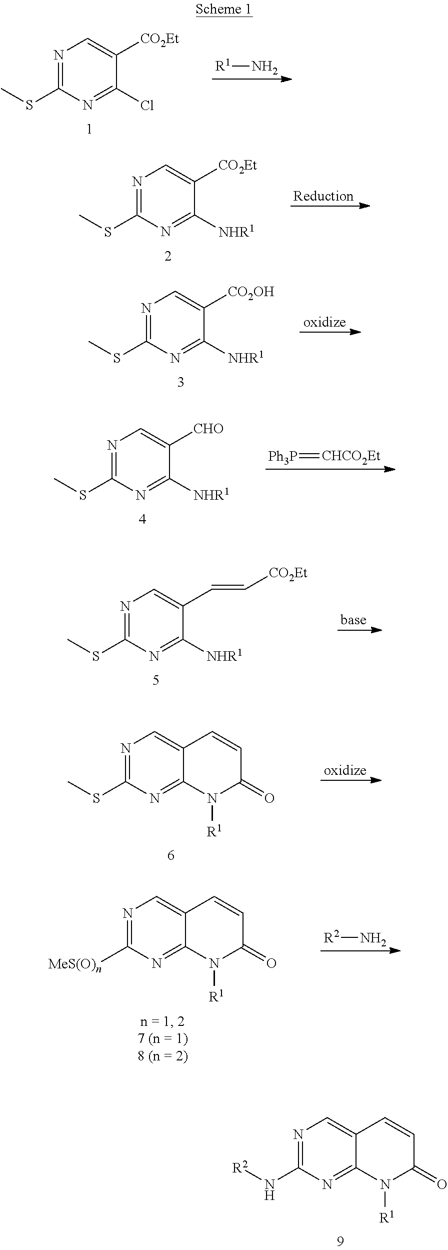

When the first step of the synthesis is performed with ammonium hydroxide as shown in Scheme 2, compound 14 can be made similarly as that of compound 6 as shown in Scheme 1. Intermediate 14 can be oxidized with oxaziridine to form 15 and/or 16, which then can react with an aromatic amine to produce analogue 17, which in turn was alkylated at N8 by treatment with sodium hydride and an alkyl halide to produce a desired product 18. Alternatively, compound 14 was alkylated at N8 to give structure 6, then oxidized to & and/or 8, which is then treated with amines to produce the desired product 18.

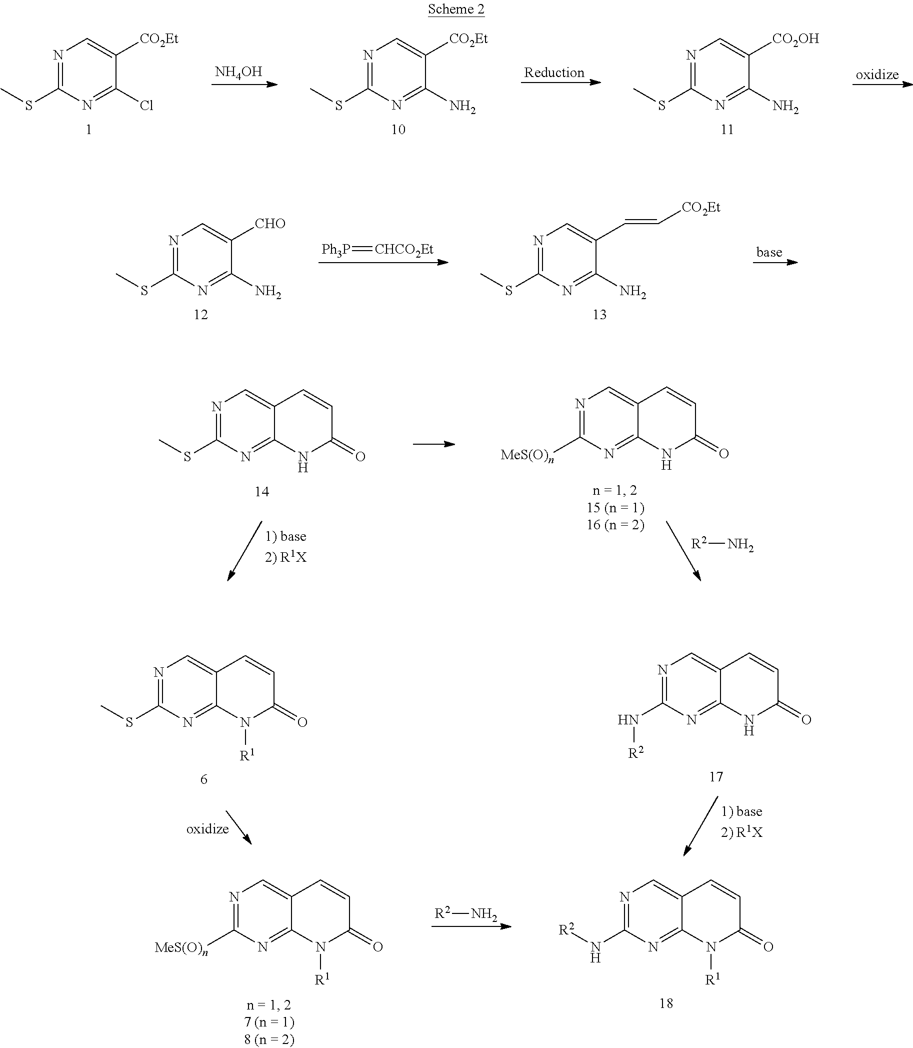

### Example 1

Examples of compounds can be prepared by some of these routes and are detailed below. One of the examples of the synthesis of PS004 is shown in Scheme 3 below.

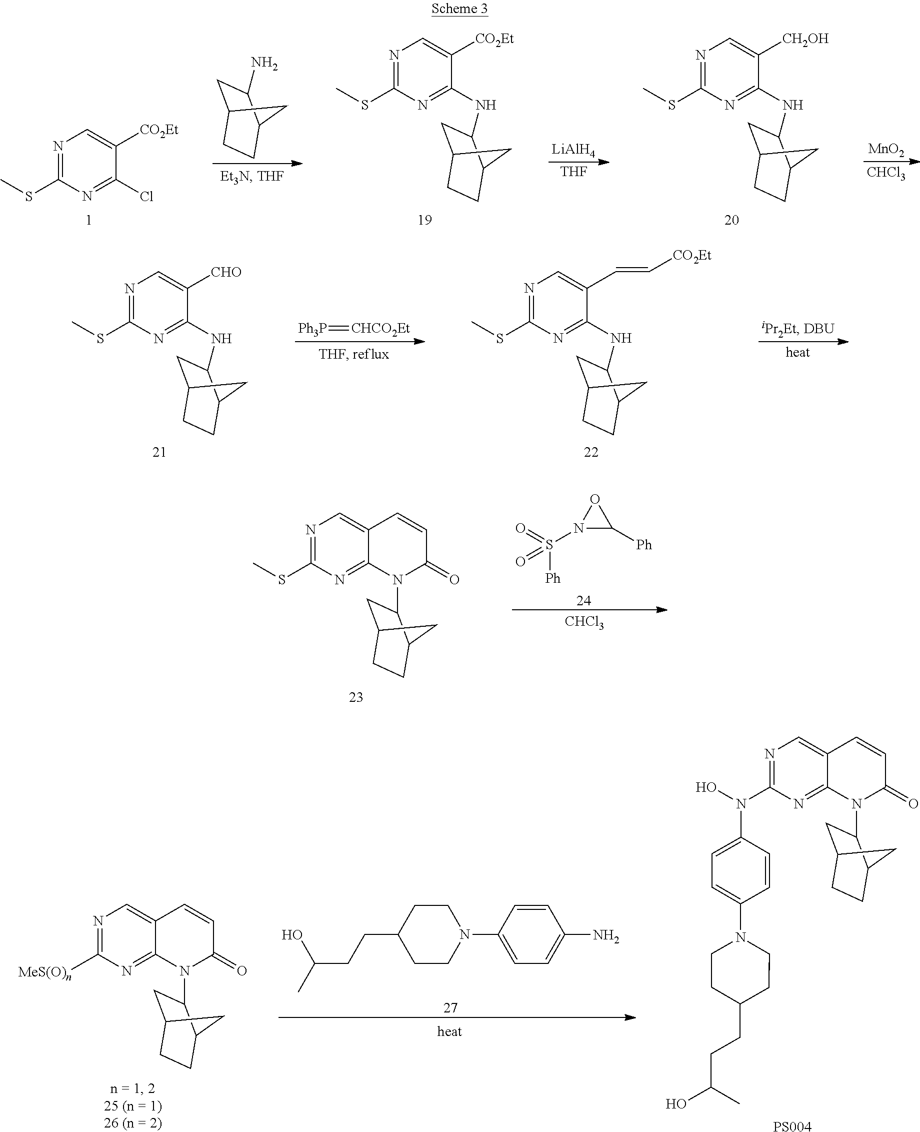

### Step 1: Preparation of Compound 19.

To a room-temperature solution of 4-chloro-2-methanesulfanylpyrimidine-5-carboxylic acid ethyl ester (1) (1 eq.) in tetrahydrofuran (about 0.3 M) is added triethylamine (3 eq.) followed by (1S,2S,4R)-bicyclo[2.2.1]heptan-2-amine (excess amount). The solution is stirred for about 30 minutes, then concentrated in vacuo, and partitioned between chloroform and saturated aqueous sodium bicarbonate. The organic layer is dried over magnesium sulfate, filtered, and concentrated to provide compound 19, which is directly used in the next step without further purification.

### Step 2: Preparation of Compound 20.

A solution of compound 19 (1 eq.) in tetrahydrofuran (about 0.4 M) is added dropwise to a room temperature suspension of lithium aluminum hydride (1.6 eq.) in tetrahydrofuran. After about 10 minutes, the reaction is carefully quenched with water and 15% NaOH, and the mixture is stirred for about 1.5 h. The white precipitate is removed by filtration, washing with ethyl acetate. The filtrate is concentrated in vacuo and 1:1 hexane:ethyl acetate is added. The solids are collected to give compound 20.

### Step 3: Preparation of Compound 21.

To compound 20 (1 eq.) in chloroform (about 0.05 M) is added manganese oxide (about 7 eq.). The suspension is stirred at room temperature for about 2 h and an additional of manganese oxide (about 2 eq.) is added. Stirring is continued for about 4-5 h. The mixture is filtered through Celite, washing with chloroform. The filtrate is concentrated in vacuo to give compound 21.

### Step 4: Preparation of Compound 22.

To a room-temperature solution of compound 21 (1 eq.) in tetrahydrofuran (about 0.3 M) is added (carbethoxymethylene)triphenylphosphorane (1.3 eq.). The reaction mixture is heated at reflux for about 70 minutes. The reaction mixture is concentrated in vacuo, and the residue is purified by flash chromatography, eluting with ethyl acetate, to provide compound 22;

### Step 5: Preparation of Compound 23.

To a room-temperature solution of compound 22 (1 eq.) in N,N-diisopropylethylamine (7 eq.) is added 1,8-diazabicyclo[5.4.0]undec-7-ene (1.15 eq.). The reaction mixture is heated at reflux overnight then cooled to room temperature. The resultant solid was collected by filtration and washed with 1:1 hexane:ethyl acetate to give compound 23. The filtrate is concentrated in vacuo and upon the addition of hexane, a solid formed that is collected, washed with hexane, and purified by flash chromatography eluting with ethyl acetate to provide an additional amount of title product 23.

### Step 6: Preparation of Compound 25.

To a room-temperature solution of compound 23 (1 eq.) in chloroform (0.1 M) is added (+/−)-trans-2-(phenylsulfonyl)-3-phenyloxaziridine (1.2 eq.). The solution is stirred at room temperature overnight then concentrated in vacuo. The residue is treated with ethyl acetate to give a solid that is collected by filtration and washed with ethyl acetate to provide compound 25.

### Step 7: Preparation of PS004.

To compound 25 (1 eq.) is added more than 1 equivalent of amine 27, the reaction mixture is heated at 100° C. to 175° C. for less than 1 h. A typical workup involves dilution of the cooled reaction mixture with ethyl acetate followed by an aqueous wash with a sodium bicarbonate solution. The organic layer is dried over magnesium sulfate, filtered, then evaporated to dryness. The crude product is purified by crystallization from ethyl acetate and hexanes, or by silica gel chromatography to give the desired product PS004.

Other analogous compounds, such as PS002, PS003, PS005, PS006, PS007, PS016, and other compounds described herein can be made similarly using, for example, some of the optionally substituted reagents listed below, either commercially available or synthesized using available synthetic methods in the art. In some cases, protection and de-protection of certain groups of the reagents are needed during the synthesis.

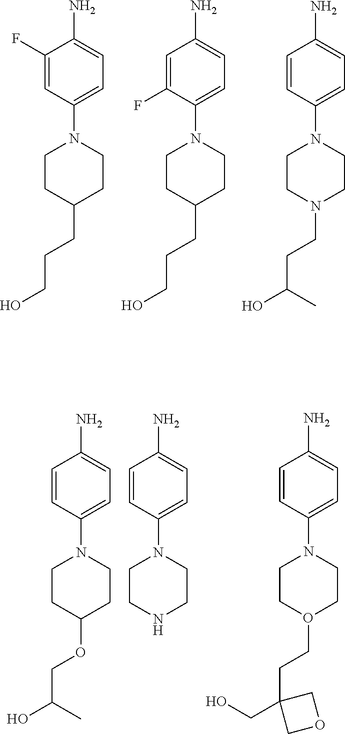

For example, one of the above aromatic amines can be prepared as shown in Scheme A below or can be purchased commercially. In this case, the Boc-protected substituted aniline (tert-butyl 4-(4-aminophenyl)piperazine-1-carboxylate) is used as a reagent during the synthesis, and the Boc-protecting group is removed after the synthesis. Other substituted anilines can be prepared similarly using appropriate reagents.

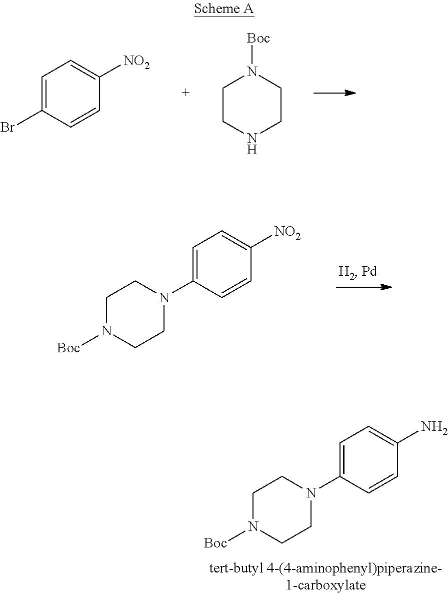 [SMILES: O=[N+]([O-])c1ccc(Br)cc1 ; CC(C)(C)OC(=O)N1CCNCC1 ; CC(C)(C)OC(=O)N1CCN(c2ccc([N+](=O)[O-])cc2)CC1 ; CC(C)(C)OC(=O)N1CCN(c2ccc(N)cc2)CC1]

In some embodiments, the pyrido[2,3-d]pyrimidin-7-one analogues described herein are prepared by the general routes illustrated in Schemes 4, 5, and 6. As shown in Scheme 4, compound 29 can be prepared from compound 4 which can be made according to the method described in Scheme 1 by treating with reagent 28. The hydroxy group of compound 29 can be oxidized to a ketone to form compound 30. Oxidation of the methyl sulfide in compound 30 with an oxaziridine provides the corresponding sulfoxide or sulfone, which in turn can be replaced by an amine under heating condition to form compound 31. Performing Wittig, Homer-Wadsworth Emmons, Knoevenagel or related chemistry such as enolate anion chemistry on the ketone at the C₅ position of a substituted 4-aminopyrimidine 31 with the treatment of reagent 32 can install the C₅-C₆ double bond of the pyrido[2,3-d]pyrimidinone ring system (positions referring below).

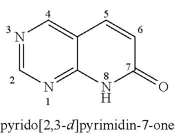 [SMILES: O=c1ccc2cncnc2[nH]1]

These reactions proceed under conditions that would be well known to one skilled in the art, with the employment of a suitable base such as NaH, NaOEt, LDA, BuLi, HMDS and the like. Ring closure to form the desired product 33 typically occurs spontaneously under the reaction conditions when the double bond geometry is such that the pyrimidine ring and the ester group are placed in a cis-relationship across the newly formed double bond. Otherwise gentle warming in a suitable organic solvent to a temperature less than 100° C. may be required to promote ring closure. When the double bond geometry is such that the pyrimidine and the ester are placed in a trans relationship across the double bond, ring closure can be driven by double bond isomerization, for example by heating in DBU to a temperature between 100° C. and 200° C., or by treatment with a radical source such as iodine and UV light under conditions that would be well known to one skilled in the art. The order of ring formation and side chain installation may be reversed similar to that shown in Schemes 5 below.

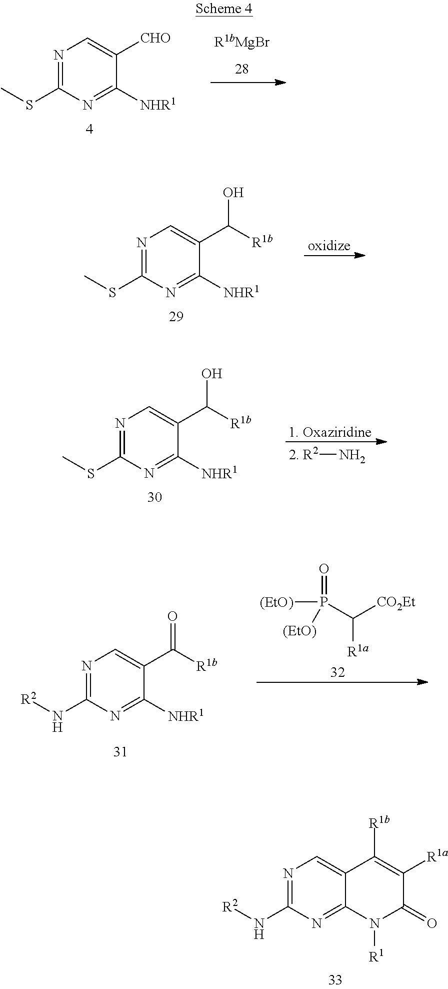

Alternatively, synthesis of compounds of the instant disclosure as shown in Scheme 5 may proceed through substituted 2-chloro-pyrimidine intermediate 34, which can be made using methods known in the art. Compound 35 can be made via Wittig, Homer-Wadsworth Emmons, Knoevenagel reaction of the ketone at the C₅ position of 34 with reagent 32 followed by spontaneously ring closure as described above. Installation of the C₂ side chain of compound 35 typically proceeds with catalysis by [(t-Bu)₂P(OH)]₂PdCl₂ (POPd), Pd(OAc)₂ or Pd₂dba₃ and a suitable ligand, such as BINAP, Xantphos or a related phosphine-based Pd ligand to generate the desired product 33.

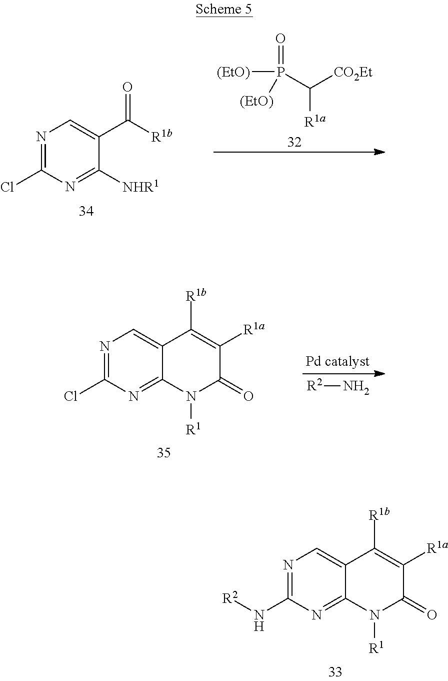

Another alternative route to prepare compounds of the instant disclosure as shown in Scheme 6 may proceed through intermediate 31, which can be made as described above. Treating compound 31 with reagent 36 via Wittig, Homer-Wadsworth Emmons, Knoevenagel reaction and spontaneous ring closure can afford the pyrido[2,3-d]pyrimidinone 37 without a substituent at C₆ position. Introducing Br at C₆ position of 37 can be achieved by treatment of NBS to form 38. A Stille coupling reaction can be performed on compound 38 using reagent 39 and a catalyst to form compound 40, which can be treated with HCl to form a ketone substituent at C₆ position to give the desired product 41. The Stille reactions in Schemes 6 are typically performed under palladium catalysis using reagents such as Pd(OAc)₂, Pd₂(dba)₃, or Pd(PPh₃)₄, and PdCl₂(PPh₃)₂. Typical solvents include dimethoxyethane, tetrahydrofuran, acetonitrile and toluene which may be warmed during the reaction to temperatures in the range of 100-200° C.

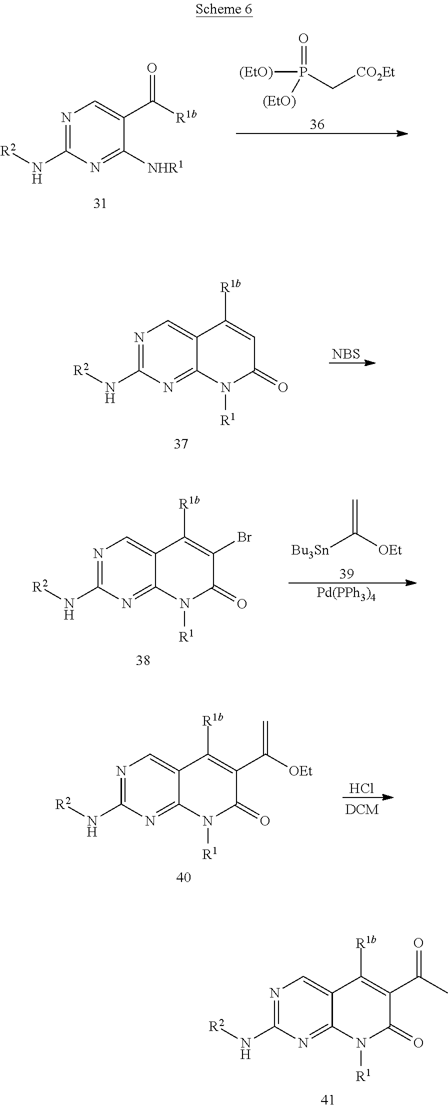

### Example 2

Examples of compounds can be prepared by some of these routes described above. One of the examples of the synthesis of PS009 is shown in Scheme 7 below.

Starting with the commercially available compound 1, replacing Cl with amine 42 gives compound 43. The ester group of 43 is then reduced to a hydroxy group forming 44. The hydroxy group in 44 is then oxidized to an aldehyde group forming 45. The aldehyde in 45 is then converted to a ketone forming 48 via a 2-step synthesis. Compound 48 can be converted to 49 via Wittig, Homer-Wadsworth Emmons, Knoevenagel reaction and spontaneous ring closure. After introducing Br at C₆ position of 49 to form 50, the methyl sulfide group of 50 is then oxidized with reagent 24 to form sulfoxide and/or sulfone, which is then replaced by amine 53 to form 54. Compound 54 can be converted to 55 via a Stille coupling with reagent 39 in the presence of a palladium catalyst such as Pd(PPh₃)₄. Finally, the treatment of 55 with HCl generates the desired compound PS009.

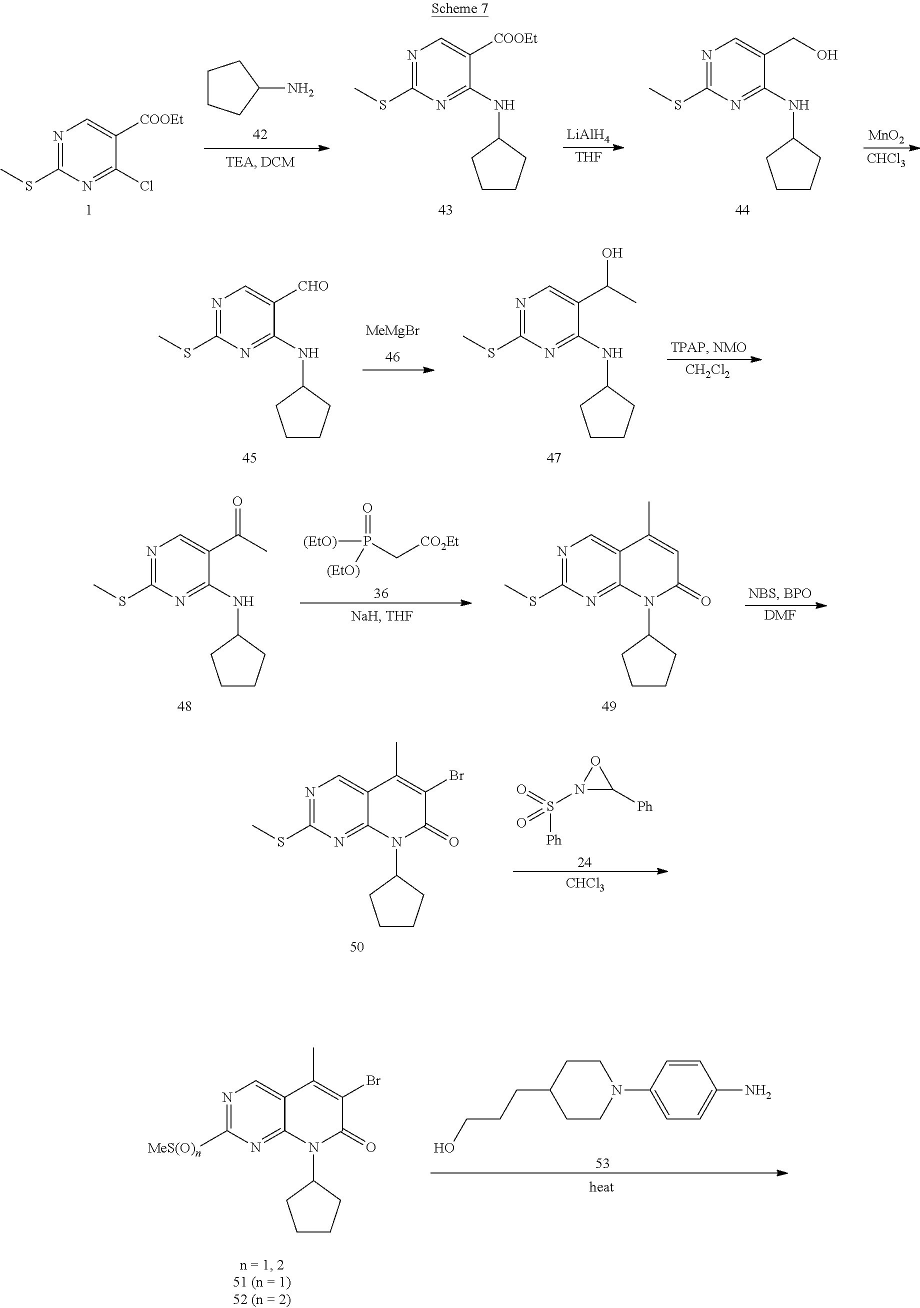

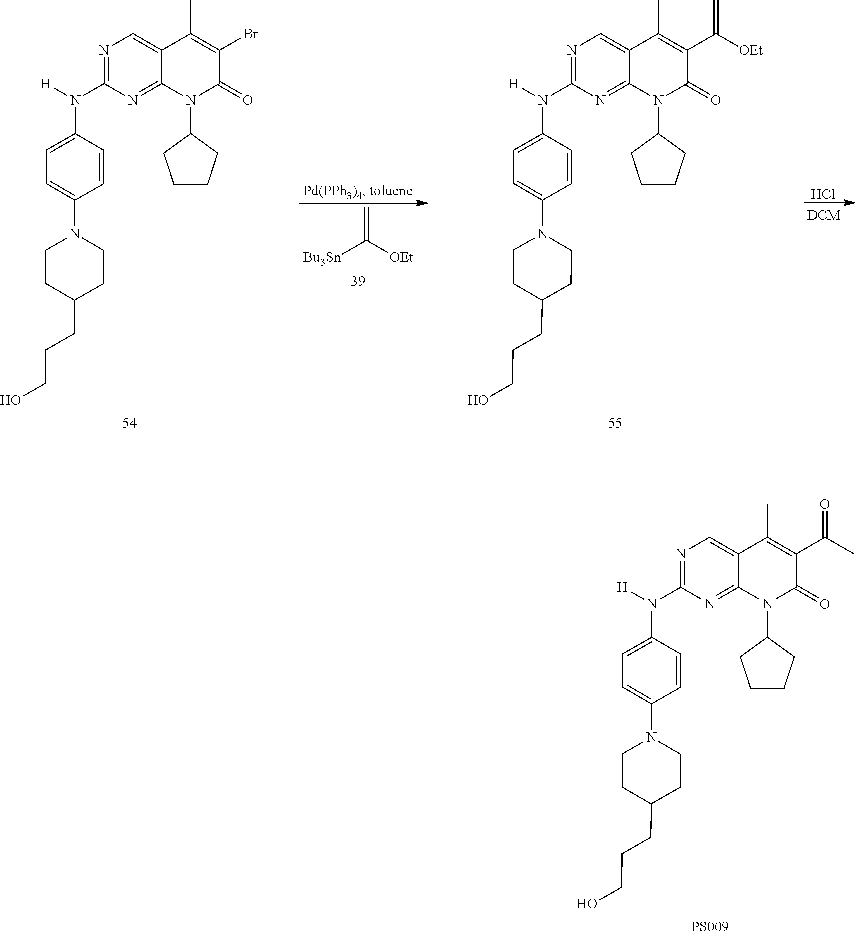

Other compounds described herein, such as PS008, PS010, can be made similarly using the method described in Scheme 7 using other appropriate reagents. In some cases, protection and de-protection of certain groups of the reagents are needed during the synthesis.

### Biological Assays and Test Results:

### Cell Lines and Cell Culture

The following human tumor cell lines, all Retinoblastoma (Rb)-proficient, were used in this study: MDA-MB231 (breast cancer), MDA-MB453 (breast cancer), U87MG (glioblastoma) and H460 (lung cancer). MDA-MB468 (breast cancer) and SW1783 (glioblastoma) Rb-deficient cells were also included in the study, as well as the MCF10A non-transformed mammary epithelial cell line. Tumor cell lines were maintained in DMEM or RPMI-1640 medium, depending on the cell line, supplemented with 10% FBS. MCF10A cells were grown in complete mammary epithelial growth medium (MEGM, Lonza). Cell lines were authenticated by short tandem repeat (STR) loci profiling with the GenePrint® 10 System (Promega).

### Kinase Profiling

The kinase profiling of compounds using 27 protein kinases were performed. For each compound, 200 μL of stock solution in 100% DMSO (5 mM) were provided as a 100× stock solution of the highest concentration to be used in IC₅₀ determination (50 μM). Ten semi-logarithmic dilutions were stepwise performed, and tested in singlicate against a panel of 27 kinases: AKT1, CDC7/DBF4, CDK1/CycA2, CDK1/CycB1, CDK1/CycE1, CDK12/CycK, CDK19/CycC, CDK2/CycA2, CDK2/CycE1, CDK3/CycC, CDK3/CycE1, CDK4/CycD1, CDK4/CycD3, CDK5/p25NCK, CDK5/p35NCK, CDK6/CycD1, CDK6/CycD3, CDK7/CycH/MAT1, CDK8/CycC, CDK9/CycK, CDK9/CycT1, DYRK1A, DYRK2, ERK1, HIPK2, PCTAIRE1/CycY, PIM1. The IC₅₀ values are summarized in Table 1 below. Lower IC₅₀ correlates with higher binding affinity between the kinases and the compounds.

TABLE 1
The IC₅₀ (μM) for the compound binding affinity with 27 protein kinases.

| | PD- | | | | | | | |
|---|---|---|---|---|---|---|---|---|
| Kinase | 0183812 | PS002 | PS003 | PS004 | PS005 | PS006 | PS007 | PS008 |
| AKT1 aa1-480 | 48.04 | >50 | >50 | >50 | >50 | >50 | 11.65 | >50 |
| CDC7/DBF4 | 18.76 | 14.58 | 7.607 | 9.902 | 1.534 | 5.569 | 0.5374 | >50 |
| CDK1/CycA2 | 28.75 | 34.04 | 7.855 | 14.47 | 0.1797 | 4.277 | 0.2085 | 39.08 |
| CDK1/CycB1 | 32.09 | 42.1 | 4.686 | 16.37 | 0.04067 | 1.957 | 0.0973 | 0.7215 |
| CDK1/CycE1 | 8.093 | 14.56 | 2.971 | 3.317 | 0.1732 | 1.213 | 0.04084 | 3.037 |
| CDK12wt/CycK | 3.163 | 28.39 | 3.176 | 0.9226 | 0.03081 | 1.014 | 0.02579 | 28.08 |
| CDK19/CycC | 17.41 | 24.94 | 19.24 | 9.598 | 0.2338 | 4.131 | 0.2885 | 25.74 |
| CDK2/CycA2 | 0.3411 | 19.69 | 0.8103 | 0.4516 | 0.01299 | 0.203 | 0.01707 | 3.522 |
| CDK2/CycE1 | 0.1223 | 7.291 | 0.5454 | 0.3034 | 0.01718 | 0.1085 | 0.007803 | 3.641 |
| CDK3/CycC | 5.213 | 27.07 | 3.954 | 2.101 | 0.05413 | 0.8849 | 0.04012 | 1.862 |
| CDK3/CycE1 | 0.8854 | 17.16 | 1.132 | 0.654 | 0.02719 | 0.3065 | 0.01189 | 25.9 |
| CDK4/CycD1 | 0.0316 | 1.266 | 0.1496 | 0.03534 | <1.5E−03 | 0.01887 | <1.5E−03 | 0.09892 |
| CDK4/CycD3 | 0.07178 | 3.836 | 0.3764 | 0.1231 | 0.002968 | 0.04664 | 0.002253 | 0.2612 |
| CDK5/p25NCK | 30.06 | >50 | 4.66 | 4.414 | 0.07253 | 1.458 | 0.0523 | 9.435 |
| CDK5/p35NCK | 1.841 | 38.57 | 1.921 | 1.675 | 0.03467 | 0.7706 | 0.0159 | 3.685 |
| CDK6/CycD1 | 0.01988 | 0.4847 | 0.07316 | 0.02735 | <1.5E−03 | 0.01248 | <1.5E−03 | 0.01949 |
| CDK6/CycD3 | 0.3009 | 12.62 | 0.623 | 0.442 | 0.01685 | 0.1078 | 0.009659 | 0.6721 |
| CDK7/CycH/MAT1 | 45.36 | 40.73 | 19.96 | 21.69 | 0.07234 | 7.998 | 0.0911 | 11.75 |
| CDK8/CycC | 0.2555 | 0.1221 | 0.6768 | 0.1617 | 0.01715 | 0.1032 | 0.3066 | 27.29 |
| CDK9/CycK | 0.1742 | 7.046 | 0.4557 | 0.141 | 0.01014 | 0.1485 | 0.007166 | 0.06747 |
| CDK9/CycT1 | 0.4844 | 8.072 | 0.9176 | 0.264 | 0.02137 | 0.2643 | 0.01355 | 0.1749 |
| DYRK1A | 31.91 | >50 | >50 | >50 | 1.377 | 46.77 | 1.181 | 11.75 |
| DYRK2 | >50 | >50 | >50 | 40.45 | 4.724 | >50 | 2.314 | 9.242 |
| ERK1 | 29.57 | 47.5 | >50 | 37.82 | 4.534 | 8.321 | 2.753 | 0.9494 |
| HIPK2 | >50 | >50 | >50 | >50 | 6.06 | 14.7 | 5.565 | 5.577 |
| PCTAIRE1/CycY | 43.46 | >50 | 43.84 | 13.53 | 0.00769 | 3.845 | 0.004036 | 35.86 |
| PIM1 | 35.86 | 27.95 | >50 | >50 | 7.186 | >50 | 0.4854 | >50 |

| Kinase | PS009 | PS010 | PS016 | Palbociclib | Ribociclib | Abemaciclib | Roscovitine |
|---|---|---|---|---|---|---|---|
| AKT1 aa1-480 | >50 | >50 | >50 | >50 | >50 | >50 | >50 |
| CDC7/DBF4 | >50 | 13.55 | 1.979 | 25.86 | 0.3178 | >50 | 42.08 |
| CDK1/CycA2 | >50 | 4.206 | 1.356 | >50 | 4.517 | >50 | 12.14 |
| CDK1/CycB1 | 32.54 | 2.054 | 0.6141 | >50 | 1.315 | >50 | 3.846 |
| CDK1/CycE1 | 19.73 | 1.445 | 0.5269 | 42.94 | 3.965 | >50 | 5.523 |
| CDK12wt/CycK | 17.42 | 0.7989 | 0.3388 | 17.19 | 0.4955 | 21.01 | 3.257 |
| CDK19/CycC | 36.38 | 2.849 | 2.786 | 11.81 | 0.3616 | >50 | 30.37 |
| CDK2/CycA2 | 0.7679 | 0.2217 | 0.04974 | 6.943 | 0.1067 | 28.72 | 1.26 |
| CDK2/CycE1 | 2.942 | 0.2765 | 0.07165 | 11.14 | 0.8196 | >50 | 0.4406 |
| CDK3/CycC | 7.663 | 0.1705 | 0.5545 | 6.868 | 0.3227 | 49.69 | 11.56 |
| CDK3/CycE1 | 17.07 | 0.7003 | 0.09335 | 21.44 | 1.66 | >50 | 1.559 |
| CDK4/CycD1 | 0.02599 | 0.003147 | 0.008858 | 0.004118 | <1.5E−03 | 0.008647 | 24.6 |
| CDK4/CycD3 | 0.05541 | 0.005481 | 0.02085 | 0.01033 | 0.0052 | 0.01873 | 33.9 |
| CDK5/p25NCK | 4.853 | 0.3954 | 0.6525 | 15.24 | 2.189 | >50 | 1.971 |
| CDK5/p35NCK | 1.858 | 0.1132 | 0.2546 | 5.958 | 0.6411 | >50 | 0.9728 |
| CDK6/CycD1 | 0.01302 | 0.001827 | 0.007422 | 0.002043 | 0.002547 | 0.008568 | 30.98 |
| CDK6/CycD3 | 0.1252 | 0.02121 | 0.03326 | 0.02396 | 0.08385 | 0.2993 | >50 |
| CDK7/CycH/MAT1 | 30.75 | 2.027 | 2.37 | >50 | 7.352 | >50 | 1.789 |
| CDK8/CycC | >50 | 16.75 | 0.03786 | >50 | 2.02 | >50 | 38.51 |
| CDK9/CycK | 0.05598 | 0.01199 | 0.04685 | 0.4217 | 0.01658 | 0.4316 | 1.57 |
| CDK9/CycT1 | 0.1935 | 0.0366 | 0.09253 | 1.264 | 0.05181 | 1.126 | 2.068 |
| DYRK1A | >50 | 3.653 | 28.79 | 14.39 | 0.01447 | >50 | 35.49 |
| DYRK2 | >50 | 1.258 | 19.71 | 6.909 | 0.0139 | >50 | 38.07 |
| ERK1 | 48.95 | 22.1 | 10.68 | 12.84 | >50 | >50 | 25.74 |
| HIPK2 | >50 | 0.1869 | >50 | 14.01 | 0.03975 | 46.37 | >50 |
| PCTAIRE1/CycY | >50 | 4.313 | 2.018 | 41.7 | 0.5889 | >50 | >50 |
| PIM1 | >50 | 8.139 | 40.06 | 34.04 | 0.02443 | >50 | >50 |

Note:
PD-0183812, Palbociclib, Ribociclib, Abemaciclib, and Roscovitine are reference compounds for comparison.

As shown in Table 1, most of the compounds tested exhibited high affinity for CDK4 and CDK6 and several of them (such as PS008, PS009 and PS016) showed additional affinity for CDK1/2. PS006, PS010, and PS016 also presented affinity for CDK5. PS005 and PS007 seem to bind many other CDK family members.

### Cell Culture, GI₅₀ and Cell Proliferation Analysis

All human cancer cell lines (Table 2) were obtained from the American Type Culture Collection, and were maintained in DMEM (Hyclone) or RPMI-1640 medium (Sigma) supplemented with 10% fetal bovine serum (Sigma). Non-transformed MCF10 cell line was maintained in MEGM Mammary Epithelial Cell Growth Medium (Lonza). Immortalized mouse embryonic fibroblasts (MEFs) were maintained in DMEM with 10% fetal bovine serum.

TABLE 2
Human cancer cell lines

| Cell Line | Tissue Type | Growth Medium |
|---|---|---|
| MDA-MB-231 | Breast carcinoma cell line | DMEM-10% FBS |
| MDA-MB-468 | Breast carcinoma cell line | DMEM-10% FBS |
| BT-549 | Breast carcinoma cell line | RPMI-10% FBS |
| MCF-7 | Breast carcinoma cell line | DMEM-10% FBS |
| ZR75-1 | Breast carcinoma cell line | RPMI-10% FBS |
| T47D | Breast carcinoma cell line | RPMI-10% FBS |
| U87MG | Glioblastoma cell line | DMEM-10% FBS |
| SW1783 | Glioblastoma cell line | DMEM-10% FBS |
| NCI-H460 | Lung carcinoma cell line | RPMI-10% FBS |
| MCF-10A | Mammary epithelial cell line | MEGM |
| Wild-type MEFs | Immortalized MEFs | DMEM-10% FBS |
| Cdk2_KO MEFs | Immortalized MEFs | DMEM-10% FBS |
| Cdk4/6_KO MEFs | Immortalized MEFs | DMEM-10% FBS |
| Cdk2/4/6_KO MEFs | Immortalized MEFs | DMEM-10% FBS |

Proliferation Assays: Determination of GI₅₀ Values

To determine the GI₅₀ (Growth inhibition 50%), cells were seeded in 96-well plates at 20-40% confluence (10,000-20,000 cells/well as previously optimized for each cell line), and treated with inhibitors at 11 concentrations ranging from 10 μM to 0.033 μM. 48 h later cells were fixed with PFA 4% for 15 minutes, stained with 10 μg/ml Hoechst 33342 (Molecular Probes, Thermo Fisher) for 30 minutes and washed twice with PBS. Cells were imaged in a high content screening system (Opera Phoenix™, Perkin Elmer) using a 20× dry objective (30 fields/well). Cell counts were determined by the Opera software (Perkin Elmer) and data were further processed with SPSS software. GI₅₀ was calculated by estimating the absolute IC₅₀ using dose-response inhibition tool in Prism6 (Graphpad Software Inc.).

Cdk-deficient MEFs were plated in 10-cm dish in triplicate (100,000/well) and treated with the selected compounds at the indicated concentration, based on GI₅₀ previously determined in the MDA-MB-231 cell line. Cells were counted in an optical microscope 3 and 6 days after treatment to estimate the relative cell growth in each condition.

For a detailed comparison of the antiproliferative effects of PS009 and Palbociclib, seven breast cancer cell lines were treated with both inhibitors at their respective GI₅₀ (determined in the MDA-MB-231 cell line as a reference) during a 6-day period. Relative cell proliferation was calculated by counting cell number at day 6 in treated cells versus cells treated with vehicle (DMSO).

### The Subject Compounds Suppress Proliferation of Human Cancer Cell Lines

The cell growth inhibitions of the subject compounds were tested in multiple human cancer cell lines with wild-type Retinoblastoma protein: MDA-MB-231 (breast), U87-MG (glioblastoma), H460 (lung); and MCF10 (as an example of non-transformed human cells) and mutant cells. Table 3 summarizes the growth inhibitory concentration required to reduce 50% of proliferation (GI₅₀) of the compounds tested in this set of human cancer cell lines. As shown in Table 3, most subject compounds suppressed cell proliferation with a potency similar to the known clinically relevant CDK4/6 inhibitors, such as, palbociclib, ribociclib, abemaciclib, RO-3306, or PD-0183812 (reference compounds), with the exception of PS002.

TABLE 3
GI₅₀ (μM) in different human cancer cell lines

| | MDA- | | | |
|---|---|---|---|---|
| | MB231 | U87MG | H460 | MCF10 |
| Palbociclib | 0.74 | 0.43 | 1.32 | 0.47 |
| Ribociclib | 0.7 | 0.26 | 1.47 | 0.54 |
| Abemaciclib | 1.5 | 0.47 | 3.1 | 1.24 |
| RO-3306 | 3.2 | >50 | 25.6 | 2.8 |
| PD-0183812 | 2.15 | 7.45 | 1.57 | 3.3 |
| PS002 | 35.2 | >50 | 18 | 34.6 |
| PS003 | 3.9 | 4.1 | 1.6 | 7.5 |
| PS004 | 3.6 | 4.5 | 1.4 | 1.43 |
| PS005 | 0.14 | 0.05 | 0.13 | 0.05 |
| PS006 | 1.4 | 2.34 | 0.19 | 0.42 |
| PS007 | 0.15 | 0.04 | 0.16 | 0.07 |
| PS008 | 4.5 | 2.44 | 2.57 | 0.7 |
| PS009 | 3.0 | 0.9 | 1.55 | 1.17 |
| PS010 | 0.5 | 0.16 | 0.37 | 0.08 |
| PS016 | 1.23 | 1.12 | 0.44 | 0.13 |

Note:
n = 3 independent experiments for MDA-MB-231 and U87-MG cell lines; and n = 2 independent experiments for H460 and MCF10 cell lines.

### Cell Cycle Analysis

Cells were grown in 6-cm dishes and treated for the indicated compounds at the GI₅₀ concentration for 24 h. Cells were collected by trypsinization, washed with PBS and fixed with cold 70% ethanol. Cells were treated with 250 μg/ml RNase (Qiagen) for 30 minutes at 37° C. and stained with 10 μg/ml Propidium Iodide (Sigma). Cell cycle was analyzed by flow cytometry using a LSR Fortessa Analyzer. Cell cycle profiles were generated and analyzed with FlowJo software.

### Flow-Cytometry

Cells were collected by tripsinization at several times after treatment with the indicated compounds and, then, fixed with cold 70% Ethanol. DNA content was determined by staining with propidium iodide (10 μg/ml, Sigma Aldrich). Data acquisition was performed with a LSR Fortessa analyzer (BD Biosciences) and analysed using FlowJo software.

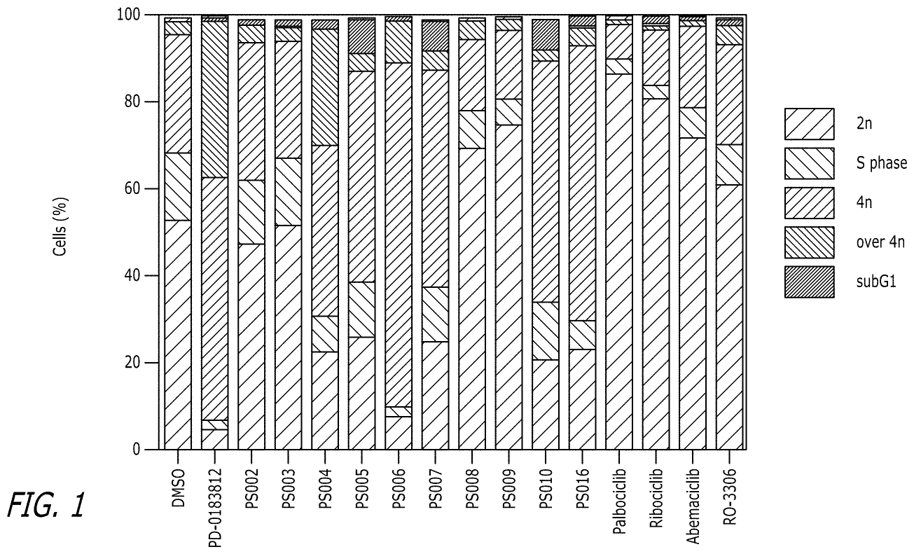

Current CDK4/6 inhibitors suppress proliferation by preventing cells from entering S phase. Cell cycle analyses for DNA content revealed a robust G0/G1-phase arrest in cells treated with palbociclib, ribociclib or abemaciclib, consistent with suppression of CDK4/6 activity (FIG. 1). PD-0183812 on the other hand, arrested cells with a 4N DNA content suggesting G2/M arrest or mitotic defects leading to tetraploidy. PS008 and PS009 induced accumulation of cells in G0/G1, similar to CDK4/6 specific inhibitors, whereas other PS compounds such as PS003, PS006 or PS016 induced G2/M arrest as detected by 4N DNA content. PS004, PS005, PS007 and PS010 exhibited a mixed phenotype, leading to accumulation of cells both in G1 and G2/M phases. Some compounds such as PS005, PS007 and PS010 led to cell death as indicated by increased sub-G1 accumulation (FIG. 1).

### Senescence Assays

MDA-MB-231 cells were seeded in 6-well plates (65,000 cells/well) and treated with the selected compounds at their respective GI₅₀. At 3, 7 and 14 days after treatment cells were stained with Senescence β-Galactosidase Staining Kit (Cell Signaling) at 37° C. overnight. Blue coloured senescent cells were counted with an optical microscope. Medium and compounds were refreshed every 3 days.

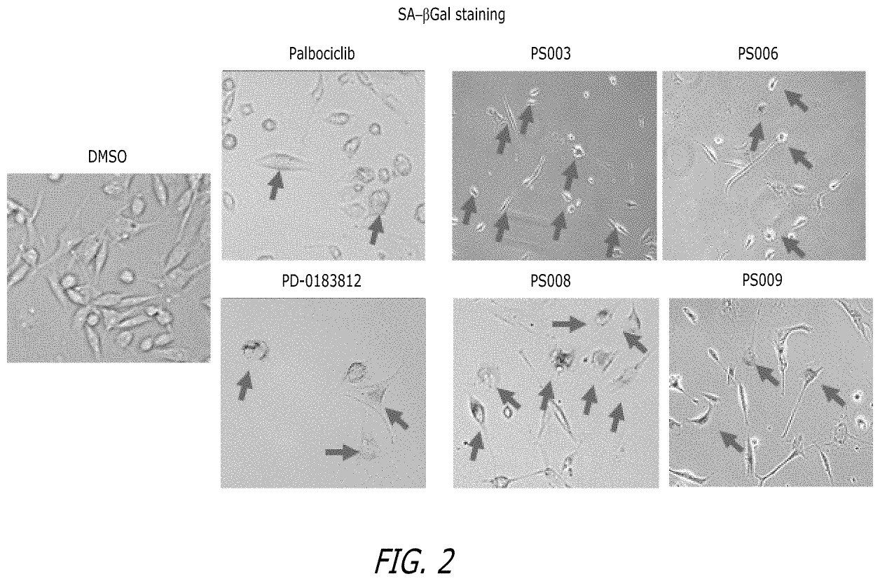

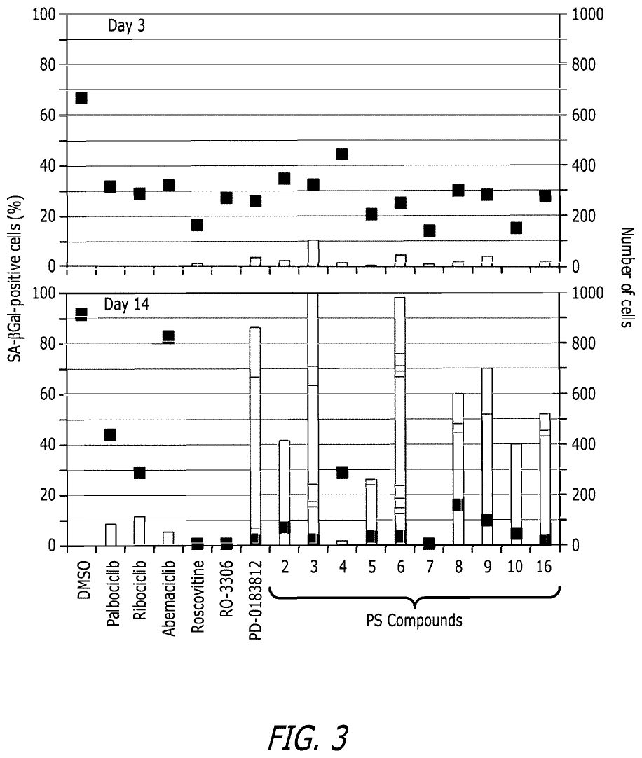

Cell cycle arrest can be irreversible in case of senescence induction. Cellular senescence is defined by several non-exclusive features including flat cell morphology, positive staining for senescence-associated beta-galactosidase at pH 6.0 (SA-(βGAL), DNA damage, and a specific secretory phenotype. In order to test senescence induction by the subject compounds, we stained MDA-MB-231 cells for SA-βGAL after short-term (3 days) or long-term (14 days) exposure to the different inhibitors. As illustrated in FIGS. 2 and 3, PD-0183812, PS003, PS006, PS008, PS009, among others, induced high levels of SA-βGAL staining indicative of senescence with higher efficiency when compared to reference CDK4/6 inhibitors.

### Antibodies and Immunoblotting

MDA-MB-231 and MDA-MB-468 cells were treated with the indicated compounds using the G150 corresponding to the MDA-MB-231 cell line, and 48 hours later lysed in Laemmli buffer (60 mM Tris-Cl pH 6.8, 10% Glycerol, 2% SDS). Protein concentration was determined using BCA method (Pierce). Whole-cell lysates (25 μg) were separated on TGX Criterion 4-15% Bis-Tris acrylamide gels (BioRad), transferred to nitrocellulose membranes (BioRad), and probed using the following specific antibodies: β-actin from Sigma Aldrich; phospho-histone H₃ (Ser10) from Millipore; Retinoblastoma from BD Pharmingen; phospho-Rb (S807/811) and Cyclin B from Cell Signaling, and Cyclin A, p21 and FOXM1 from Santa Cruz Biotechnology.

### The Subject Compounds do not Exclusively Depend on CDK4/6 Activity

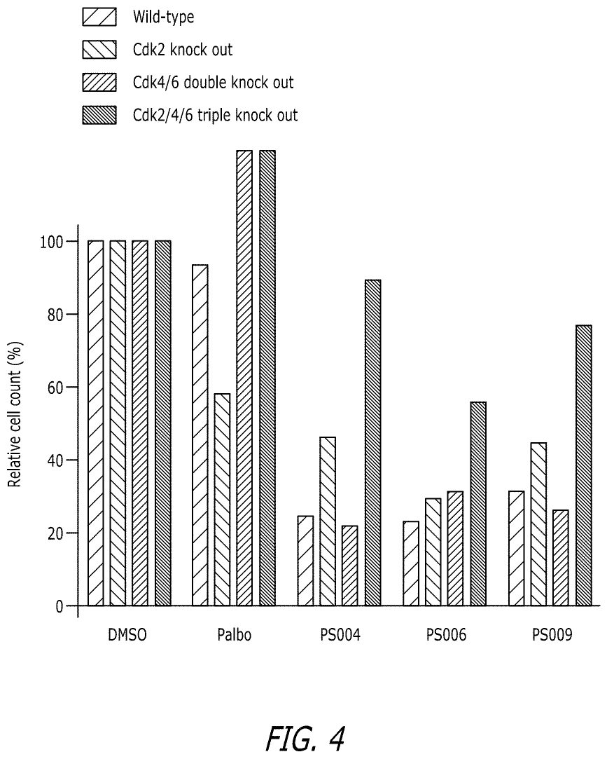

As shown in Table 1 above, most compounds bind CDK4/6 with high affinity in addition to other CDKs including CDK2. To further investigate the relative functional dependence of the subject compounds on CDK2 versus CDK4 or CDK6, the specific effects of PS004, PS006 and PS009 were tested on CDK-deficient mouse embryonic fibroblasts (MEFs) obtained from Cdk2 knock out, Cdk4; Cdk6-double knock out and Cdk2; Cdk4; Cdk6-triple knock out mouse models. FIG. 4 shows the relative cell number of MEF cultures after exposure to the different compounds. Reference compound Palbocilib did not reduce cell proliferation in Cdk4; Cdk6-double knock cells, suggesting a strong dependency on CDK4/6 activity (green columns in FIG. 4) to exert its anti-proliferative effects. The subject compounds, however, efficiently prevented cell proliferation of Cdk4; Cdk6-null cells (green columns), and to certain extent that of Cdk2; Cdk4; Cdk6-triple mutant cells (purple columns), suggesting certain dependence on the activity of CDK2 and perhaps other related kinases.

CDK4/6 kinases drive cell cycle progression by phosphorylating the retinoblastoma protein (pRB), thus, releasing its repressive activity on transcription. Accordingly, CDK4/6 specific inhibitors are inefficient in pRB-deficient cells, as cell cycle transcription is induced with independence of CDK4/6 activity. The subject compounds were therefore evaluated to what extent depend on the presence of a functional pRB. Two pairs of human cancer cell lines representing pRB-wild-type and pRB-deficient breast cancer and glioblastoma were tested. As expected, palbociclib exhibited a significant increase in GI₅₀, suggesting reduced efficacy, when comparing pRB mutant cells with those harboring a functional pRB, both in brain cancer (19.9 vs. 5.7 μM) and breast cancer cells (9.5 vs. 1.7 μM) as shown in Table 4 below. PS004 was also more inefficient in pRB-deficient glioblastoma cells, but showed similar effect when comparing pRB-null and pRB-wild-type breast cancer cells. Finally, PS006 and PS009 were equally efficient in pRB-null or pRB-proficient brain and breast cancer cells, suggesting no dependence on the presence of this tumor suppressor. In agreement with these data, DNA content analysis showed efficient arrest of pRB-deficient tumor cells in G2/M (4N) cells by most compounds, suggesting that these subject compounds could target other G0/G1-independent activities in cells in which the G1 checkpoints are abrogated.

TABLE 4
Growth inhibition (GI₅₀, μM) of palbociclib
and selected compounds in pRB-wild
type and pRB-mutant cancer cell lines.

| | | | Breast cancer | |
|---|---|---|---|---|
| | Glioblastoma | | pRB | |
| | pRB | | Wild- | pRB |
| | Wild- | pRB | Type | Mutant |
| | Type | Mutant | MDA- | MDA- |
| | U87MG | SW1783 | MB-231 | MB-468 |
| | (n = 4) | (n = 4) | (n = 4) | (n = 2) |
| Palbociclib | 5.7 | 19.9 | 1.7 | 9.5 |
| PS004 | 5.5 | 11.5 | 3.5 | 3.0 |
| PS006 | 2.2 | 2.2 | 1.7 | 2.5 |
| PS009 | 6.9 | 8 | 3.3 | 3.9 |

Note:
n = 4 independent experiments for U87-MG, SW1783 and MDA-MB-231 cells; and n = 2 independent experiments for MDA-MB-468 cells.

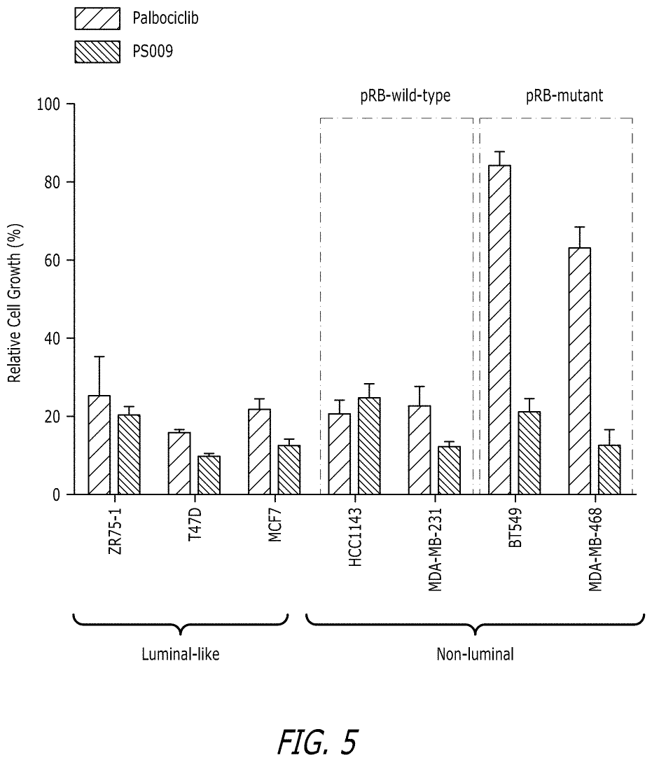

Palbociclib is known to exert a more potent anti-proliferative effect in luminal-like cells when compared to non-luminal breast cancer cell lines, likely due to the presence of active pRB signaling in luminal cells, thereby inducing a stronger dependence on CDK4/6 activity. Therefore, it was tested to see whether the spectrum of inhibition in a panel of breast cancer cell lines was wider for the subject compounds when compared to specific known CDK4/6 inhibitors. FIG. 5 shows the relative cell growth of a panel of human breast cancer cell lines, both luminal-like (ZR75-1, T47D and MCF7) and non-luminal (HCC1143, MDA-MB-231, BT549, MDA-MB-468) after 6 days of treatment with palbociclib or PS009. Whereas two out of the four non-luminal cells lines were resistant to palbociclib, in agreement with mutation of pRB in these cells, PS009 was able to efficiently inhibit both luminal and non-luminal breast cancer cells, including pRB-mutant non-luminal cells, confirming that PS009 has a broader spectrum than palbociclib, a known CDK4/6 inhibitor, and PS009 does not depend on a functional pRB pathway to exert its antiproliferative effects.

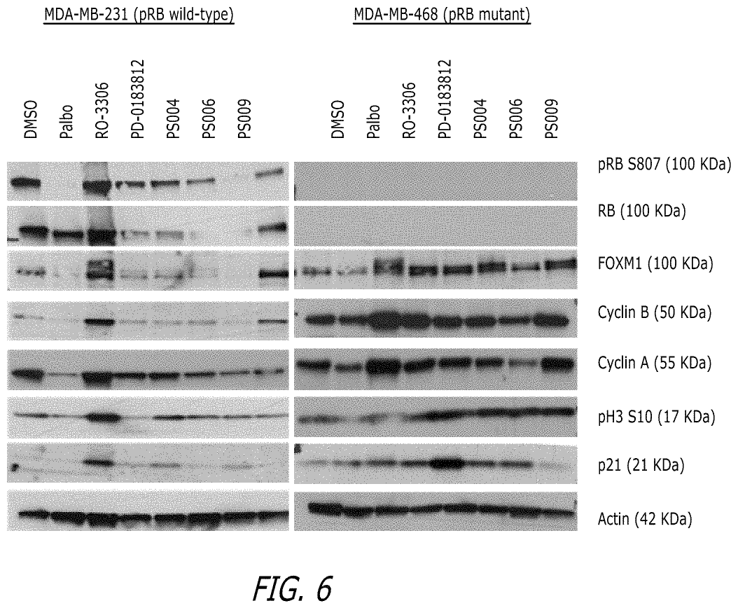

Additionally, the levels of different proteins involved in cell cycle progression, such as Cyclins A and B, FOXM1 and the cell cycle inhibitor p21^{cip1}, as well as the phosphorylation status of pRB (Serine 807) and Histone H₃ (Serine 10), were biochemically evaluated. As shown in FIG. 6, treatment with PS009 and palbociclib (or “Palbo” in short) resulted in strong inhibition of pRB phosphorylation and reduced levels of most cell cycle markers, whereas these defects were less dramatic with the other compounds.

### In Vivo Evaluation of Selected Subject Compounds

For histological analysis, mice tissues and human xenografts were fixed in 10%-buffered formalin (Sigma) and embedded in paraffin wax. Sections of 3- or 5-μm thickness were stained with haematoxylin and eosin. Additional immunohistochemical examination was performed using a specific antibody against phospho-Rb (Ser807/811; Cell Signaling) or Ki67 (DAKO).

### Tumor Xenografts

Athymic nude mice (6-week-old females provided by Harlan Laboratories/ENVIGO), were injected subcutaneously in both flanks with 5×10⁶ MDA-MB-231 cells in 100 μl PBS-0.1% glucose. Approximately two weeks after injection, when tumors reached 100 mm³, mice were randomized in 6 different treatment groups (8 mice/group): DMSO (vehicle for PS compounds), PS004, PS006, PS009, Lactate Buffer (vehicle for palbociclib) and palbociclib. DMSO, PS004, PS006 and PS009 were diluted in sesame oil and administered intraperitoneally twice per week during two weeks at 100 mg/Kg. Palbociclib was dissolved in lactate buffer (50 mM sodium lactate, pH4) and delivered by oral gavage at 100 mg/Kg 5 days/week, following by two days of drug-holiday, during 12 days. Tumor growth was monitored using caliper measurements and tumor size was estimated using the following formula: Tumor volume (mm³)=d²·D/2 where d and D, are the shortest and longest diameter in mm, respectively. After completing the treatment, mice were sacrificed and tumors were extracted, weighed, and fixed for histological analysis.

### Statistical Analysis

Statistical analysis was carried out using Prism 6 (Graphpad Software Inc.). All statistical tests of comparative data were done using two-sided, unpaired Student's t-tests or ANOVA for differential comparison between two groups or more groups, respectively. Data with p<0.05 were considered statistically significant (*, p<0.05; **, p<0.01; ***, p<0.001).

### Efficacy of Subject Compounds

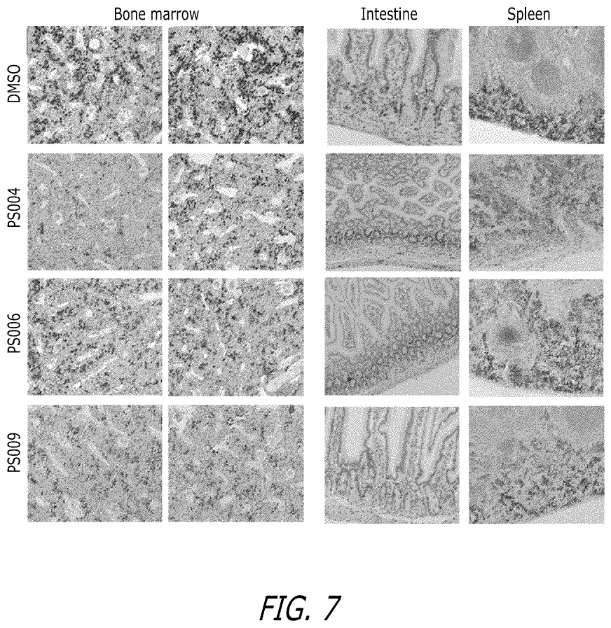

For in vivo evaluation, the target engagement for the subject compounds in vivo was tested first. The phospho-RB staining in bone marrow, intestine and spleen showed an evident RB target inhibition in vivo, especially after treatment with PS004 and PS009 (FIG. 7).

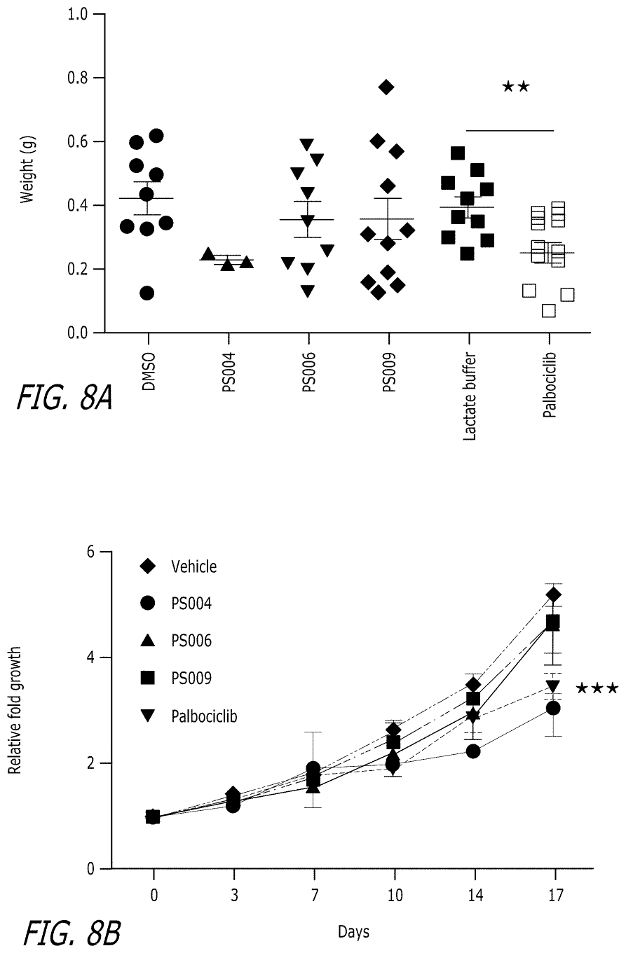

The therapeutic effect of the subject compounds was then tested by treating mice previously injected subcutaneously with MDA-MB-231 breast cancer cells. Palbociclib (oral administration) was used as a reference and subject compounds PS004, PS006 and PS009 were injected intraperitoneally once tumors were evident. The dose amount of 100 mg/kg dosage in these cases was administered at a frequency of two injections per week for the total of two weeks of treatment. During the two-week treatment, subject compound PS004 and reference compound palbociclib significantly reduced both tumor growth and tumor weight compared to their respective controls (FIGS. 8A and 8B), while compounds PS006 and PS009 did not exert any antitumoral effect as determined by general tumor fold growth and final tumor weight.

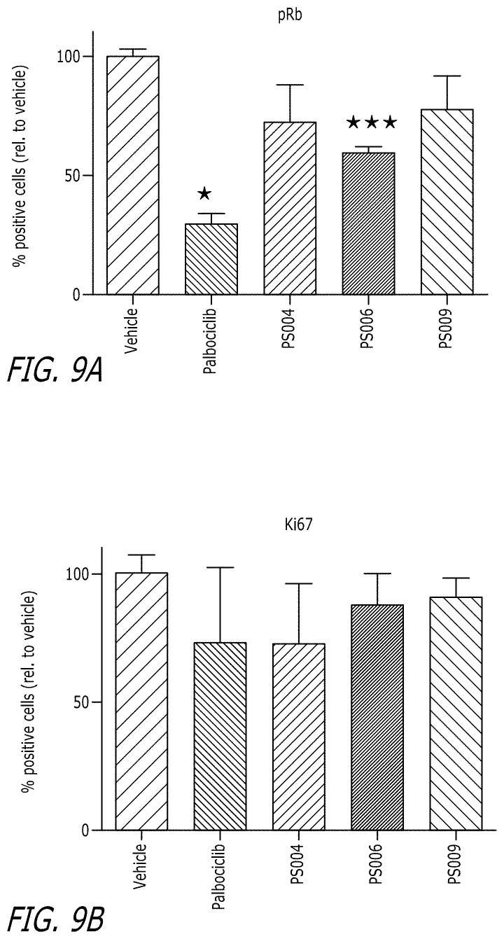

Biomarker analysis revealed that both PS006 and palbociclib significantly reduced phospho-RB levels in the xenografts, while PS004 and PS009 also led to the reduction of phospho-Rb phosphorylation which however did not reach statistical significance (FIG. 9A). Additionally, Ki67 levels slightly diminished upon treatment with PS004 or palbociclib, although these data did not reach statistical significance (FIG. 9B).

### Solubility and Stability of Subject Compounds

Given the general low or lack of activity observed in vivo for some of the subject compounds tested, the solubility and stability of these compounds were tested in vivo. As shown in Table 5, while Palbociclib is largely soluble and stable in vivo, the subject compounds exhibit clear defects in their chemical formulation to sustain either good solubility or stability in vivo. In particular, PS006 and PS009 seem to be highly unstable and insoluble, which could explain their low activity in vivo.

Therefore, the low activity or lack thereof observed in vivo for some of the subject compounds tested is believed due to their low solubility, and/or low stability. Thus, the therapeutic evaluation of such compounds in vivo in their current formulations is difficult. It is also believed that other formulations, such as a combination with a cyclodextrin, may improve solubility, and/or stability of the subject compounds, which could result in favorable or better potency in vivo.

TABLE 5
Analysis of solubility, metabolic stability and permeability of palbociclib and selected subject compounds.

| | Solubility (pH 7.4) | | Metabolic stability (60-90 min test: | | | | | | |
|---|---|---|---|---|---|---|---|---|---|
| | Thermodynamic | Kinetic | Clearance and t_{1/2}) | | | | | | |
| | solubility | solubility | Human | Mouse | Human | Mouse | Permeability Caco-2 cells | | |
| Compound | (μM) | (μM) | microsomes | microsomes | hepatocytes | heptocytes | Permeability | Efflux | Summary |
| Palbo | 2.32 | 19.94 | 23.6 | 18.3 | <6.4 | 14.4 | 0.4 | 50.78 | Soluble, |
| | | | μl/min/mg | μl/min/mg | μl/min/10⁵ | μl/min/10⁵ | (low) | (likely) | Stable, Low |
| | | | (58.8 min) | (75.7 min) | (>216.8 min) | (96.5 min) | | | permeable/ |
| | | | | | | | | | Efflux |
| PS004 | <2.00 | <1.00 | 40.8 | 54.5 | <6.4 | 9.7 | <0.44 | N.D.** | Insoluble, |
| | | | μl/min/mg | μl/min/mg | μl/min/10⁵ | μl/min/10⁵ | (low) | | Stable, Low |
| | | | (34 min) | (25.4 min) | (>216.8 min) | (143.4 min) | | | permeable |
| PS006 | <2.00 | <1.00 | 119 | 184.7 | 49 | 75.7 | 4.62 | 0.59* | Insoluble, |
| | | | μl/min/mg | μl/min/mg | μl/min/10⁵ | μl/min/10⁵ | (high) | (poor) | Unstable, High |
| | | | (11.6 min) | (7.5 min) | (28.3 min) | (18.3 min) | | | permeable/No |
| | | | | | | | | | efflux |
| PS009 | <2.00 | <1.00 | 33 | 46.9 | 41.7 | 67.8 | <0.43 | N.D.** | Insoluble, |
| | | | μl/min/mg | μl/min/mg | μl/min/10⁵ | μl/min/10⁵ | (low) | | Stable? Low |
| | | | (42 min) | (29.5 min) | (33.3 min) | (20.4 min) | | | permeable |

**N.D. = not detected.

### Toxicologic Evaluation In Vivo

For toxicity studies, athymic nude mice (6-week-old females provided by Harlan Laboratories/ENVIGO), were treated with vehicle (DMSO) or the PS compounds (PS004, PS006 and PS009). Stock solutions were diluted in sesame oil at 10 mg/ml and administered intraperitoneally at two doses (50 or 100 mg/Kg) twice per week during a period of 2 weeks (3 mice/group). After treatment mice were sacrificed, weighed and several tissues (lungs, intestine, bone marrow and spleen) were extracted and fixed in 10%-buffered formalin for histological examination. Blood counts were analyzed in a differential hematology analyzer (Abacus, Diatron).

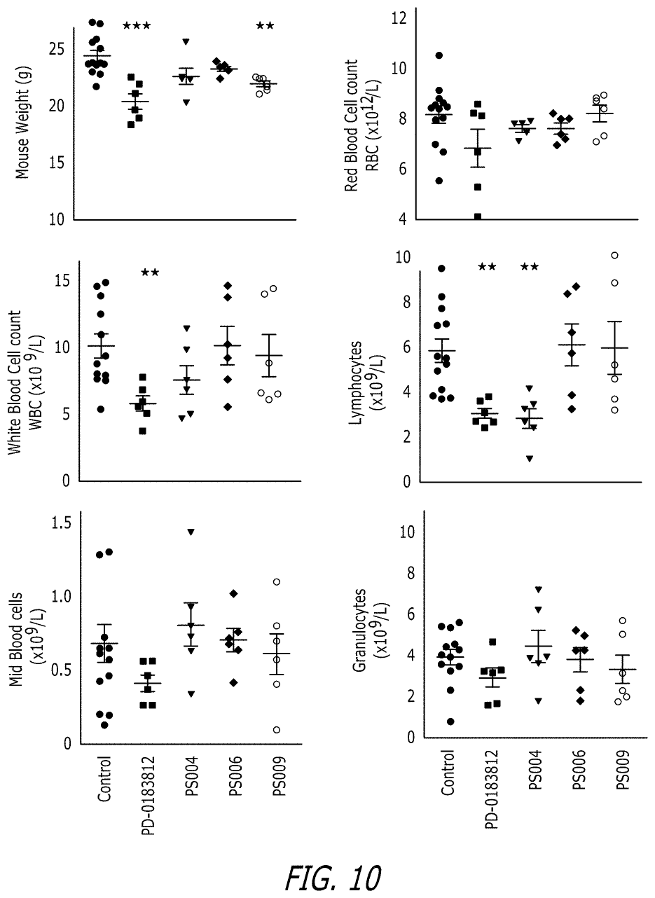

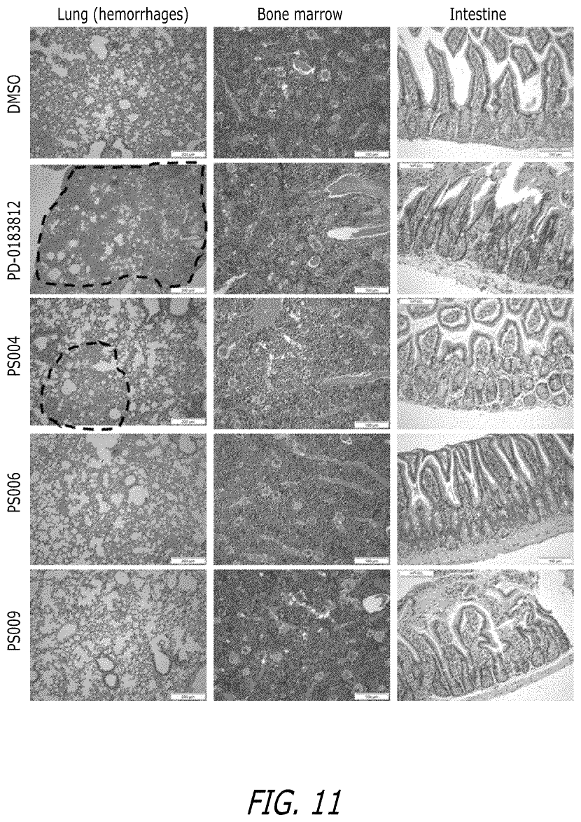

To evaluate the toxicity of selected compounds, the healthy female athymic nude mice was first injected intraperitoneally with PS004, PS006 and PS009, as well as PD-0183812 (50 mg/kg or 100 mg/kg each) twice a week for two weeks, and then measured different parameters at the endpoint. Reference compound PD-0183812 induced significant weight loss when compared to control DMSO, as well as reduced levels of total white blood cells or lymphocytes (FIG. 10). PS009 induced weight loss but no defects in blood counts, whereas PS004 resulted in reduced lymphocyte counts without other obvious alterations. Both PD-0183812 (6 out of 6 mice) and, to a lesser extent, PS004 (4 out of 6 mice) induced lung toxicity which induced abundant hemorrhage in the lungs (FIG. 11). PS006 and PS009 induced lung hemorrhages with edema in 1 out of 6 mice tested for each compound. Further histopathological analysis revealed also aberrant mitotic figures in the intestine and hypoplasia/reactive bone marrow in PD-0183812 and PS004-treated mice, as well as active extramedullar hematopoiesis in the spleen of mice treated with PD-0183812, PS004 or PS006.

### The Advantage of the Subject Compounds

Inhibition of CDK4/6 has significant therapeutic potential in hormone-positive HER2-negative advanced breast cancer, and three specific inhibitors have been recently approved for clinical use. However, these inhibitors are relatively inefficient in hormone-negative breast tumors, and their putative use in other tumor types is still under pre-clinical or clinical evaluation. As the CDK family is composed by 20 different kinases, and compensatory roles are expected between different family members, there may be limited application for the inhibitors that specifically inhibit CDK4/6.

The series of the subject compounds described herein display relative specificity against CDK4/6 kinases and significant potency in vitro, similar to that achieved by known CDK4/6 inhibitors such as palbociclib. In addition, some of the subject compounds are able to bind other related CDKs such as CDK2 or CDK9 that have been proposed to have therapeutic potential in specific situations. Whereas specific inhibitors such as palbociclib and ribociclib are used in combination with hormonotherapy to treat breast cancers, abemaciclib, a CDK4/6 inhibitor that may also have activity on CDK9, is efficient in monotherapy. Among the subject compounds, PS004 inhibited cell growth with high potential arresting cells in G1/G2/M cell cycle phases, and presenting affinity not only for CDK4/6 but also for CDK2/9. This characteristic opens new unexplored mechanisms of action for CDK inhibitors, plausibly different from the commercial CDK4/6 inhibitors palbociclib, abemaciclib and ribociclib. PS006 induced high senescence compared to their counterparts, exhibited high affinity for CDK6/2/5/9 and arrested cells in G2/M phases provoking a potent cell growth inhibition. PS009 arrested cells predominantly in G1, exhibited very high SA-BGAL activity induction, and mainly bound to CDK4/6/ with also an interesting preference for CDK2 and CDK9 among other CDKs. Interestingly, several of these compounds were efficient in preventing tumor cell proliferation in pRB-mutant cells, further suggesting that their activity did not only depend on CDK4/6 kinases. In line with these observations, subject compounds PS004, PS006 and PS009 were efficient in preventing cell proliferation in Cdk4:Cdk6-double mutant, or Cdk4:Cdk6:Cdk2-triple mutant cells, whereas reference compound palbociclib did not show activity in these cells. Additionally, PS009, but not palbociclib, was able to inhibit proliferation in non-luminal, pRB-mutant breast cancer cells lines. With these unique characteristics, the subject compounds described herein may offer great therapeutic potential.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

The terms “a,” “an,” “the” and similar referents used in the context of describing the invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of any claim. No language in the specification should be construed as indicating any non-claimed element essential to the practice of the invention.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, the claims include all modifications and equivalents of the subject matter recited in the claims as permitted by applicable law. Moreover, any combination of the above-described elements in all possible variations thereof is contemplated unless otherwise indicated herein or otherwise clearly contradicted by context.

In closing, it is to be understood that the embodiments disclosed herein are illustrative of the principles of the claims. Other modifications that may be employed are within the scope of the claims. Thus, by way of example, but not of limitation, alternative embodiments may be utilized in accordance with the teachings herein. Accordingly, the claims are not limited to embodiments precisely as shown and described.

What is claimed is:

1. A compound represented by a formula: 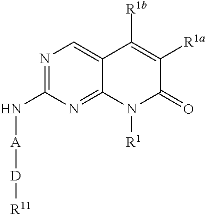
or a salt thereof;
wherein R¹ is optionally substituted C₁₋₆ alkyl or optionally substituted C₃₋₁₀ cycloalkyl;
R^{1a} is H or COCH₃;
R^{1b} is H or CH₃;
A is optionally substituted aryl or optionally substituted heteroaryl;
D is optionally substituted piperidin-1,4-yl or optionally substituted piperazin-1,4-yl;
R¹¹ is R⁸, —OR⁸, SO₂R⁸, SO₂NR⁸R⁹, COR⁸, CO₂R⁸, or CONR⁸R⁹, wherein R⁸ and R⁹ are independently H, or C₁₋₆ hydrocarbyl optionally substituted with F, Cl, Br, I, amino, OH, C₁₋₆—O-alkyl, cyano, or a C₁₋₆ geminal -alkyl-O-alkyl-.

2. The compound of claim 1, further represented by a formula: 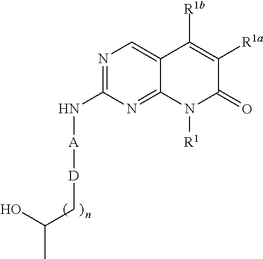
or a salt thereof;
wherein A is optionally substituted p-phenylene, or optionally substituted pyridin-2,5-yl;
wherein the 2-position attaches to NH and the 5-position attaches to D;
D is optionally substituted piperidin-1,4-yl, wherein the 1-position attaches to A;
R^{1a} is H or COCH₃;
R^{1b} is H or CH₃; and
n is 1 or 2.

3. The compound of claim 1, further represented by a formula: 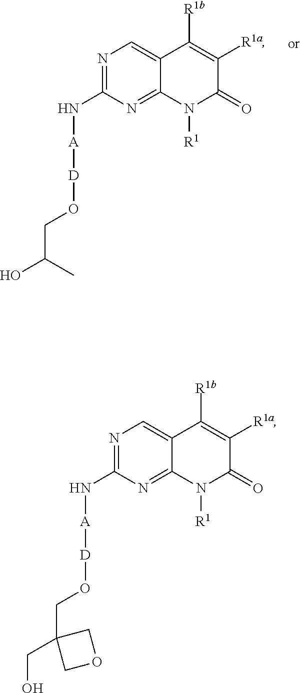
or a salt thereof;
wherein A is optionally substituted p-phenylene, or optionally substituted pyridin-2,5-yl wherein the 2-position attaches to NH and the 5-position attaches to D;
D is optionally substituted piperidin-1,4-yl wherein the 1-position attaches to A;
R^{1a} is H or COCH₃; and
R^{1b} is H or CH₃.

4. The compound of claim 1, further represented by a formula: 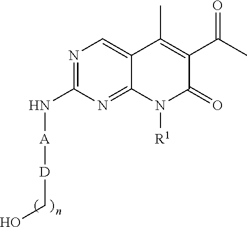
or a salt thereof;
wherein A is optionally substituted p-phenylene, or optionally substituted pyridin-2,5-yl wherein the 2-position attaches to NH and the 5-position attaches to D;
D is optionally substituted piperidin-1,4-yl wherein the 1-position attaches to A; and
n is 1, 2, or 3.

5. The compound of claim 1, further represented by a formula: 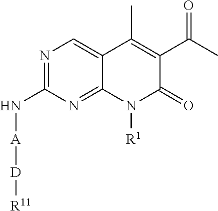
or a salt thereof;
wherein R¹ is optionally substituted bicycloheptanyl;
A is optionally substituted p-phenylene; and
D is unsubstituted piperazin-1,4-yl.

6. The compound of claim 1, further represented by a formula: 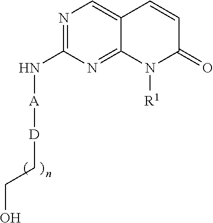
or a salt thereof;
wherein A is substituted p-phenylene, or optionally substituted pyridin-2,5-yl wherein the 2-position attaches to NH and the 5-position attaches to D;
D is optionally substituted piperidin-1,4-yl wherein the 1-position attaches to A; and
n is 1 or 2.

7. The compound of claim 1, further represented by a formula: 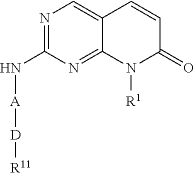
or a salt thereof;
wherein R¹ is optionally substituted bicycloheptanyl; and
A is optionally substituted pyridin-2,5-yl wherein the 2-position attaches to NH and the 5-position attaches to D, and D is optionally substituted piperazin-1,4-yl; or
A is optionally substituted phenyl and D is unsubstituted piperazin-1,4-yl.

8. The compound of claim 7, wherein R¹ is unsubstituted cyclopentanyl or unsubstituted bicyclo[2.2.1]heptanyl.

9. The compound of claim 1, wherein A is optionally substituted p-phenylene.

10. The compound of claim 1, wherein D is unsubstituted piperidin-1,4-yl.

11. The compound of claim 1, wherein D is unsubstituted piperazin-1,4-yl.

12. The compound of claim 1, wherein R¹¹ is E-Hy, wherein E is a bond, C₁₋₅ alkylene, C₁₋₅—O-alkylene, or 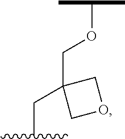
and Hy is OH or H.

13. The compound of claim 12, wherein E is optionally substituted C₁₋₅ alkylene.

14. The compound of claim 12, wherein E is —(CH₂)₃—.

15. The compound of claim 12, wherein E is —(CH₂)₂CH(CH₃)—.

16. The compound of claim 12, wherein E is —O—(CH₂)CH(CH₃)—.

17. The compound of claim 12, wherein E is

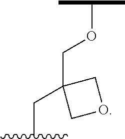

18. The compound of claim 12, wherein Hy is OH.

19. The compound of claim 12, wherein Hy is H.

20. A compound selected from: 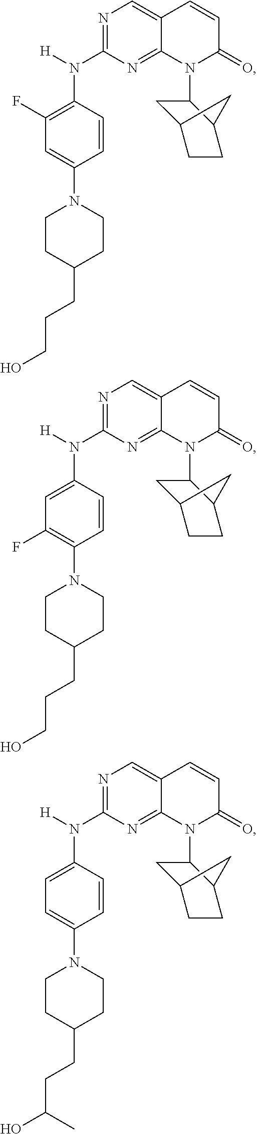 [SMILES: O=c1ccc2cnc(Nc3ccc(N4CCC(CCCO)CC4)cc3F)nc2n1C1CC2CCC1C2 ; O=c1ccc2cnc(Nc3ccc(N4CCC(CCCO)CC4)c(F)c3)nc2n1C1CC2CCC1C2 ; CC(O)CCC1CCN(c2ccc(Nc3ncc4ccc(=O)n(C5CC6CCC5C6)c4n3)cc2)CC1] 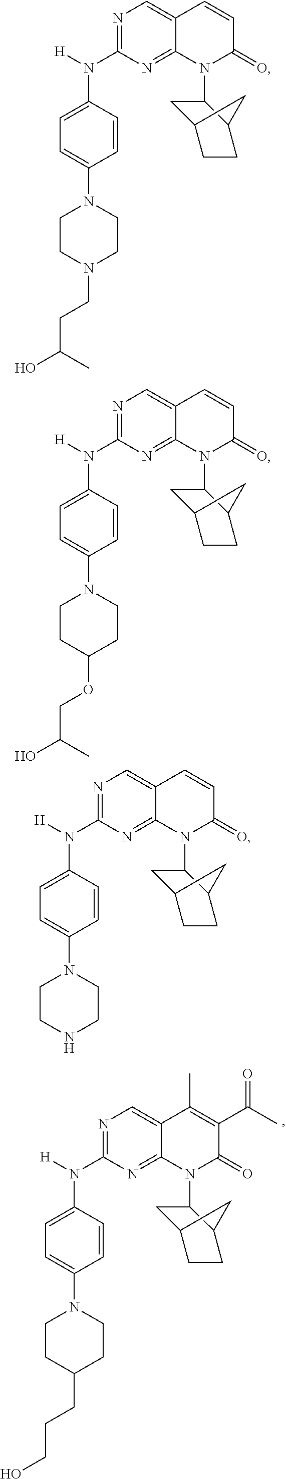 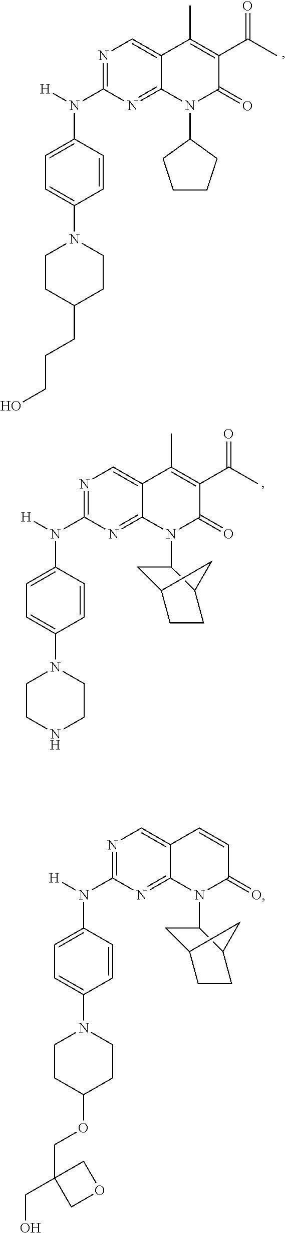 [SMILES: CC(=O)c1c(C)c2cnc(Nc3ccc(N4CCC(CCCO)CC4)cc3)nc2n(C2CCCC2)c1=O ; CC(=O)c1c(C)c2cnc(Nc3ccc(N4CCNCC4)cc3)nc2n(C2CC3CCC2C3)c1=O ; O=c1ccc2cnc(Nc3ccc(N4CCC(OCC5(CO)COC5)CC4)cc3)nc2n1C1CC2CCC1C2]
or a salt thereof.

21. A method of treating a cancer associated with a CDK inhibitor comprising administering an effective amount of a compound of claim 1.
